SEQUENCE LISTING (4 sequences)

SEQ ID NO: 1 - DNA, 20 bp, synthetic construct
  source 1..20: mol_type = other DNA
gattgacgatggatcaccag

SEQ ID NO: 2 - DNA, 20 bp, synthetic construct
  source 1..20: mol_type = other DNA
actaccagggtatctaatcc

SEQ ID NO: 3 - DNA, 67 bp, synthetic construct
  source 1..67: mol_type = other DNA
tcgtcggcagcgtcagatgtgtataagagacagcctacgggaggcagcagcctacgggnbgcascag

SEQ ID NO: 4 - DNA, 56 bp, synthetic construct
  source 1..56: mol_type = other DNA
gtctcgtgggctcggagatgtgtataagagacagtgactacnvgggtatctaatcc

(19) United States
(12) Patent Application Publication
(10) Pub. No.: US 2025/0333802 A1
(43) Pub. Date: Oct. 30, 2025

(54) METHODS OF EARLY IDENTIFICATION AND EARLY INTERVENTION FOR HIGH-RISK NEONATES WHO OTHERWISE WILL DEVELOP AUTISTIC SOCIAL IMPAIRMENTS LATER IN LIFE

(71) Applicant: The General Hospital Corporation, Boston, MA (US)

(72) Inventors: Xue-Jun Kong, Boston, MA (US); Cun-Jian Dong, Boston, MA (US)

(21) Appl. No.: 18/871,058

(22) PCT Filed: Jun. 2, 2023
(86) PCT No.: PCT/US2023/067863

§ 371 Date: Dec. 2, 2024

Related U.S. Application Data
(60) Provisional application No. 63/365,780, filed on Jun. 2, 2022.

(51) Int. Cl. C12Q 1/689; A61B 5/38; A61K 35/744; C12Q 1/686; G01N 33/573

(52) U.S. Cl. CPC C12Q 1/689; A61B 5/38; A61K 35/744; G01N 33/5735; C12Q 1/686; C12Q 2600/118; C12Q 2600/156; G01N 2800/28; G01N 2800/54

(57) ABSTRACT

Disclosed herein are methods and kits for assessing a. risk of subject developing autism spectrum disorder (ASD). The methods can include determining a level of Limosilactobacillusreuteri in a sample from the subject, or receiving results of a test indicating a level of L. reuteri in a. sample from the subject, and determining the risk of developing ASD in the subject, based at least partly on the level of L. reuteri in a sample from the subject.

### CROSS-REFERENCE TO RELATED APPLICATIONS

This application claims priority to U.S. Application No. 63/365,780, which was filed on Jun. 2, 2022, the content of which is incorporated herein by reference in its entirety.

### STATEMENT REGARDING FEDERALLY SPONSORED RESEARCH OR DEVELOPMENT

None.

### REFERENCE TO AN ELECTRONIC SEQUENCE LISTING

The contents of the electronic sequence listing (125141.04337. xml; Size: 4,610 bytes; and Date of Creation: Jun. 2, 2023) is herein incorporated by reference in its entirety.

### BACKGROUND

Autism Spectrum Disorder (ASD) has become a serious public health crisis due to 1) its rapidly rising prevalence (now 1 in 44 children in the USA), and 2) the need of lifelong personal care by caregivers for most patients despite various currently available interventions. Over two-thirds of the patients are not able to live independently due to their social and/or intellectual impairments that lead to poor daily living skills. The United States nationwide ASD-related cost is estimated to be around $223 billion in 2020 and is projected to be around $589 billion in 2030. In addition to its enormous negative impact on the public health system, ASD also poses tremendous and long-lasting economic and psychological burdens to the families of patients.

### SUMMARY

Disclosed herein are methods and kits for assessing a risk of subject developing autism spectrum disorder (ASD).

In one aspect, a method for assessing a risk of subject developing autism spectrum disorder (ASD) is provided. In embodiments, the method includes determining a level of Limosilactobacillus reuteri (L. reuteri) in a sample from the subject, or receiving results of a test indicating a level of L. reuteri in a sample from the subject. In embodiments, the method also includes determining the risk of developing ASD in the subject, based at least partly on the level of L. reuteri in a sample from the subject.

In another aspect, a kit is provided for assessing a risk of a subject developing autism spectrum disorder (ASD), wherein the subject has an age of from about one day to three years. In embodiments, the kit includes one or more reagents for detecting the presence, absence, and/or level of L. reuteri in a sample from the subject, and instructions for determining the presence, absence, and/or level of L. reuteri.

### BRIEF DESCRIPTION OF THE FIGURES

FIG. 1 is a flow chart depicting a study population distribution and procedure for the experiments described in Example 1A.

FIG. 2 is a graph showing that the baseline level of oxytocin correlated with the baseline root mean square of successive differences between normal heartbeats.

FIG. 3 is a bar graph showing that oxytocin nasal application for three months significantly increased microbiome co-occurrence networks from a previous study referenced in Example 1B.

### DETAILED DESCRIPTION

### Overview

As discussed above, ASD has become a serious public health crisis. Early diagnosis and early intervention can significantly improve the prognosis of children with ASD. However, an accurate diagnosis is usually made at 2 years of age or older (when behavioral abnormalities emerge) with the Diagnostic and Statistical Manual, Fifth Edition (DSM-5) or Autism Diagnostic Observation Schedule, Second Edition (ADOS-2).

The methods and kits disclosed herein can provide for early diagnosis and/or assessment of risk of developing ASD, in various aspects. In one or more aspects, the early diagnosis and/or assessment of risk of developing ASD can occur before the current conventional timeline for diagnosis of two years or older. Such an earlier diagnosis can allow for earlier intervention in treating ASD, which may have long term benefits. In various aspects, the early diagnosis and/or assessment of risk can occur in infants aged less than two years. As discussed in detail below, such an early diagnosis and/or assess can include determining whether a deficiency of Limosilactobacillus reuteri (L. reuteri) is present in a sample from a subject. In the same or alternative aspects, the methods may optionally also include determining responsiveness to human vocal sounds in the subject.

In various aspects, the methods disclosed herein can include prescribing or performing a therapeutic intervention designed to reduce the onset of one or more symptoms associated with ASD. In such aspects, an earlier diagnosis and/or assessment of risk followed up with earlier therapeutic intervention can be beneficial since early invention may be beneficial to patients with ASD. In certain aspects, the earlier therapeutic intervention can occur prior to two years of age.

### Definitions and Terminology

The disclosed methods and kits for assessing a risk of subject developing autism spectrum disorder (ASD) may be further described using definitions and terminology as follows. The definitions and terminology used herein are for the purpose of describing particular aspects only and are not intended to be limiting.

As used in this specification and the claims, the singular forms “a,” “an,” and “the” include plural forms unless the context clearly dictates otherwise.

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

The phrase “such as” should be interpreted as “for example, including.” Moreover, the use of any and all exemplary language, including but not limited to “such as”, is intended merely to better illuminate the invention and does not pose a limitation on the scope of the invention unless otherwise claimed.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or “B” or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use an aspect of a described embodiment or feature contained in the same, or a definitive decision to use a specific skill regarding a described embodiment or feature contained in the same. In this latter context, the modal verb “may” has the same meaning and connotation as the auxiliary verb “can.”

The terms “identical” or percent “identity,” in the context of two or more nucleic acids or polypeptide sequences, refer to two or more sequences or subsequences that are the same or have a specified percentage of amino acid residues or nucleotides that are the same (i.e., 50%, 55%, 60%, 65%, 70%, 75%, 80%, 85%, 90%, 91%, 92%, 93%, 94%, 95%, 96%, 97%, 98%, 99%, or more identity over a specified region, e.g., of an entire nucleic acid or polypeptide sequence or individual portions or domains of a nucleic acid or polypeptide), when compared and aligned for maximum correspondence over a comparison window, or designated region as measured using one of the following sequence comparison algorithms or by manual alignment and visual inspection. Such sequences are then said to be “substantially identical.” This definition also refers to the complement of a test sequence, in the context of nucleic acids.

“Autism Spectrum Disorder” refers to a complex neurological and/or developmental disorder that can be diagnosed according to standard guidelines, such as, American Psychiatric Association. Diagnostic and Statistical Manual of Mental Disorders: DSM-5. 5th ed. Washington, DC: American Psychiatric Association (2013) and/or Lord C, Rutter M, DiLavore PC, Risi S, Gotham K, Bishop S. (ADOSTM-2) Autism Diagnostic Observation Schedule, Second Edition. WPS (2012).

The term “subject” may be used interchangeably with the terms “individual” and “patient” and includes human and non-human subjects. In some aspects, subjects may be any mammal. In certain aspects, the subject is a human.

As used herein, the terms “treat” or “treatment” encompass both “preventative” and “curative” treatment. “Preventative” treatment is meant to indicate a postponement of development of a disease, a symptom of a disease, or medical condition, suppressing symptoms that may appear, or reducing the risk of developing or recurrence of a disease or symptom. “Curative” treatment includes reducing the severity of or suppressing the worsening of an existing disease, symptom, or condition. Thus, treatment includes ameliorating or preventing the worsening of existing disease symptoms, preventing additional symptoms from occurring, ameliorating or preventing the underlying systemic causes of symptoms, inhibiting the disorder or disease, e.g., arresting the development of the disorder or disease, relieving the disorder or disease, causing regression of the disorder or disease, relieving a condition caused by the disease or disorder, or stopping the symptoms of the disease or disorder,

A non-limiting example of a treatment for a subject includes, but is not limited to, administering to a subject L. reuteri.

### Methods

In various aspects, methods are disclosed for assessing a risk of subject developing autism spectrum disorder (ASD). In one or more aspects, the methods can include determining a level of Limosilactobacillus reuteri (L. reuteri) in a sample from the subject.

In various aspects, the subject can be any age. In one or more aspects, the subject can be about three years old or less, two years old or less, one year old or less, or six months old or less. In the same or alternative aspects, the age of the subject can of from one day to three years, of from one day to two years, of from one day to one year, or of from one day to six months. In certain aspects, the subject can be an infant. In some embodiments, the subject is from about one day to about three years of age.

In various aspects, the sample from the subject can be any type of sample suitable for test method being conducted. For instance, in various aspects, for determining a level of L. reuteri the sample can be a stool sample. It should be understood that additional samples may be taken from a subject for detecting other markers, for instance, as discussed further below, in certain aspects a saliva and/or blood sample may be obtained to assess for various other markers, e.g., oxytocin or the like.

Determining a level of L. reuteri can include any suitable test for identifying and/or quantifying a level of L. reuteri. In certain aspects, the level of L. reuteri can be determined using quantitative Polymerase Chain Reaction (qPCR). In various aspects, qPCR could be utilized to determine a level of L. reuteri using any suitable pair of L. reuteri species-specific PCR primer pairs. In one or more aspects, the qPCR can utilize a primer pair, where the first primer comprises the polynucleotide sequence of SEQ ID NO: 1 (GATTGACGATGGATCACCAG) and the second primer comprises the polynucleotide sequence of SEQ ID NO: 2 (ACTACCAGGGTATCTAATCC). In one or more aspects, the first primer is a polynucleotide sequence having at least 85%, at least 90%, at least 95%, or at least 99% identity to the polynucleotide sequence of SEQ ID NO: 1 and/or the first primer is a polynucleotide sequence having at least 85%, at least 90%, at least 95%, or at least 99% identity to the polynucleotide sequence of SEQ ID NO: 2. In certain aspects, the first primer can consist of the polynucleotide sequence of SEQ ID NO: 1 and/or the second primer can consist of the polynucleotide sequence of SEQ ID NO: 2. Any suitable qPCR method can be utilized. In various aspects, the qPCR method described in the Examples can be utilized.

It should be understood that determining a level of L. reuteri in the sample can include detecting and/or quantifying L. reuteri in the sample, detecting the presence of L. reuteri in the sample, and/or detecting the absence of L. reuteri in the sample.

Tetrahydrobiopterin (BH4) is a metabolite of L. reuteri. In various aspects, a level of BH4 can be determined in a sample from the subject, e.g., a stool sample. The BH4 level can be determined using any suitable quantification method, including the use of a commercially available ELISA. In certain aspects, a level of BH4 can be determined in addition to or in place of determining a level of L. reuteri as described herein.

In various aspects, the methods can include a step of determining the risk of developing ASD in the subject, based at least partly on the level of L. reuteri in the sample from the subject. In various aspects, determining the risk of developing ASD in the subject can include comparing the level of L. reuteri in the sample to a threshold value or range. In one or more aspects, the threshold may be a value or range of L. reuteri that correlates with a value or range associated with individuals who have been diagnosed with ASD. For instance, in one aspect, the threshold may be a value or range of L. reuteri that is found in individuals of similar age or withing a similar age range that were later diagnosed, e.g., at two years old, three years old, or older, with ASD according to conventional diagnostic criteria. In certain aspects, when a level of L. reuteri in the sample is similar to a threshold value and/or within a threshold value range, the subject can be determined to be at risk of developing ASD. In one or more aspects, the threshold may be a value or range of L. reuteri that correlates with a value or range associated with individuals who do not have ASD. In such an aspect, when a level of L. reuteri in the sample is outside the threshold range or value, the subject can be determined to be at risk of developing ASD.

In one or more aspects, determining the risk of developing ASD in the subject can be at least partly based on the level of L. reuteri in the sample from the subject and/or the subject's responsiveness to human vocal sounds. In various aspects, determining responsiveness to human vocal sounds can include determining a level of oxytocin in a saliva or blood sample from the subject and/or recording event-related potentials (ERP) in the subject. Levels of oxytocin in a saliva or blood sample can be determined using any suitable quantification methods, such as a commercially-available human ELISA assay. Recording event-related potentials (ERP) in the subject can be performed using any suitable methods. In certain aspects, determining responsiveness to human vocal sounds by recording ERP in the subject can be performed as described in the Examples herein.

In various aspects, determining the risk of developing ASD in the subject at least partly based on the subject's responsiveness to human vocal sounds can include comparing the results of the test(s) for the subject's responsiveness to human vocal sounds with results of other subjects diagnosed with ASD. In one or more aspects, where one or more oxytocin levels are determined, one can compare such levels to a threshold value. In such an aspect, the threshold value can be a value or range of oxytocin determined from the same or similar tests of individuals of similar age or within a similar age range that were later diagnosed, e.g., at two years old, three years old, or older, with ASD according to conventional diagnostic criteria. In certain aspects, when a level of oxytocin in the sample is similar to a threshold value and/or within a threshold value range, the subject can be determined to be at risk of developing ASD. In one or more aspects, the threshold may be a value or range oxytocin that correlates with a value or range associated with individuals who do not have ASD. In such an aspect, when a level of oxytocin in the sample is outside the threshold range or value, the subject can be determined to be at risk of developing ASD. In various aspects, where recording ERP in the subject is performed, these results can be compared to ERP recordings/results from individuals of similar age or within a similar age range that were later diagnosed, e.g., at two years old, three years old, or older, with ASD according to conventional diagnostic criteria. In such an aspect, the ERP recordings/results can be compared to one or more threshold values determined from individuals of similar age or within a similar age range that were later diagnosed, e.g., at two years old, three years old, or older, with ASD according to conventional diagnostic criteria. In various aspects, when the ERP recordings/results are similar to one or more threshold values or ranges, the subject can be determined to be at risk of ASD. In one or more aspects, the threshold value or ranges for ERP recordings/results can be based on individuals that do not have ASD. In such an aspect, when the ERP recordings/results are outside the threshold range or value, the subject can be determined to be at risk of developing ASD.

As discussed above, in various aspects, the methods disclosed herein can include prescribing or performing a therapeutic intervention designed to reduce the onset of one or more symptoms associated with ASD. In certain aspects, the therapeutic intervention can be any suitable therapy. In various aspects, the therapeutic intervention can include administering L. reuteri to the subject. In various aspects, the L. reuteri administered to the subject can be any suitable source of L. reuteri, including commercially-available strains. In one or more aspects, the L. reuteri can be L. reuteri ATCC PTA 6475, which may be obtained from BioGaia. In various aspects, L. reuteri can be administered to the subject in any manner, such as orally. In various aspects, the L. reuteri to be administered to the subject can be present in as part of a pharmaceutical composition, including one or more pharmaceutically acceptable carriers. In a preferable aspect, L. reuteri is present in sunflower seed oil when administered to the patient. In one or more aspects, L. reuteri can be administered to the subject using any suitable dosing regimen. In certain aspects, L. reuteri can be administered to the subject in a range of about 10⁶ to 10¹⁴ colony forming units (CFUs), or about 10¹⁰ CFUs. In various aspects, L. reuteri can be administered to the subject at least once a day or twice a day. In one or more aspects, L. reuteri can be administered to the subject for a period of from one week to 30 weeks, of from two weeks to 25 weeks, or of from four weeks to 16 weeks; or about one week, about two weeks, about four weeks, about eight weeks, about 12 weeks, about 16 weeks or about 20 weeks. In certain aspects, L. reuteri is administered to the subject in an amount of 3×10¹⁰ CFUs twice a day for 12 weeks.

In one or more aspects, the therapeutic intervention can additionally or alternatively include one or more conventional early intervention strategies, including but not limited to applied behavioral analysis, occupational therapy, music therapy, and communication therapy, e.g., sign language or speech therapy.

In various aspects, once a patient has begun therapeutic intervention, one or more follow-up assessments can be performed. For instance, once a patient has undergone a course of L. reuteri supplementation, a level of L. reuteri and/or BH4 can be determined from a patient's sample to assess whether or not the L. reuteri and/or BH4 are present and at what level. In the same or alternative aspects, one or more common psychometric tests can be utilized as a follow-up assessment. In various aspects, the one or more common psychometric tests can include but are not limited to the Ages and Stages Questionnaire-Third Edition (ASQ-3), Mullen Scale of Early Learning (MSEL), Vineland Adaptive Behavioral Scale Third Edition (Vineland-3), which are known to those in the field.

In various aspects, one or more metagenomic analysis of the gut microbiome of the patient may also be performed. In various aspects, such an analysis may provide information on the microbiome composition, including the abundance of L. reuteri. Any suitable method for determining a metagenomic analysis that can provide information on a gut microbiome composition may be utilized, including the example test method described in the Examples below. In one or more aspects, such a metagenomic analysis may be utilized to assess the success or failure of a therapeutic intervention, e.g., by comparing microbiome composition data before and after therapeutic intervention with supplementation of L. reuteri. In the same or alternative aspects, a metagenomic analysis can be utilized to assess a risk of a patient developing ASD. For instance, the data from a patient's metagenomic analysis, including the microbiome composition and/or abundance of L. reuteri can be compared to other individuals diagnosed with ASD and/or individuals that do not have ASD, and this comparison alone or in conjunction with the comparison of additional markers and tests described herein may be utilized to identify whether a patient is at risk of developing ASD.

### Kits

In various aspects, kits for assessing a risk of subject developing autism spectrum disorder (ASD) in a subject are disclosed. In various aspects, the subject can have an age of one day to three years. In certain aspects, the kit can include one or more reagents for detecting the presence, absence, and/or a level of L. reuteri in a sample from the subject. In one or more aspects, the one or more reagents can include any specific set of reagents suitable for determining a level of L. reuteri in a stool sample. In one example aspect, the one or more reagents can include one or more reagents for performing qPCR on the stool sample. In such and aspect, the one or more reagents can include a first primer that comprises a polynucleotide sequence having at least 85%, at least 90%, at least 95%, or at least 99% identity to the polynucleotide sequence of SEQ ID NO: 1, and a second primer is a polynucleotide sequence having at least 85%, at least 90%, at least 95%, or at least 99% identity to the polynucleotide sequence of SEQ ID NO: 2. In the same or alternative aspects, the one or more reagents can include a first primer that comprises or consists of the polynucleotide sequence of SEQ ID NO: 1, and a second primer that comprises or consists of the polynucleotide sequence of SEQ ID NO: 2.

In various aspects, the one or more reagents for detecting a level of L. reuteri in a sample from the subject can include additional qPCR reagents including a standard DNA of a known concentration of L. reuteri, and/or dilution and/or reaction buffers.

In certain aspects, the kit can include instructions for determining the presence, absence and/or level of L. reuteri in the patient sample. In such aspects, the instructions can include instructions for performing a qPCR reaction and/or instructions for determining or calculating the level of L. reuteri based on the results of the qPCR reaction. For instance, in one or more aspects, the instructions can include forming a standard curve using the qPCR data from the DNA standard control and diluted DNA standard controls, and calculating the level in the test sample using the corresponding control values with the standard curve.

In various aspects, optionally, the kit may include reagents for determining a level of BH4 in a patient's stool sample. In such aspects, the reagents can include ELISA reagents.

### EXAMPLES

The following Examples are illustrative and are not intended to limit the scope of the claimed subject matter.

Background: Social deficit is a core symptom of autism spectrum disorder (ASD). Inability to response to human vocals was found in early infancy of those later diagnosed with ASD. Probiotics Limosilactobacillus reuteri (L. reuteri) was reported to be able to rescue social deficit in several ASD mouse models with diverse etiologies via stimulating oxytocin (OT) release in the brain mediated by activation of gut vagal afferent fibers. To date, there has been no study investigated gut L. reuteri as an early biomarker or as an early intervention in human.

60 infants are to be studied at high risk for ASD (siblings of ASD patients, HRA) and 30 infants at low risk for ASD (those without a familial risk of ASD, LRA), randomly assign both groups to either the L. reuteri or placebo for 12 week intervention. Gut L. reuteri level, serum and saliva OT level, EEG/ERP cortical response to human vocals and social stimuli, autonomic function, eye tracking, microbiome profile and immunological markers will be measured through the treatment course; we will follow up these infants until they reach 2 years old and conduct standard ASD diagnosis.

Data analysis: Following the intention-to-treat principle, we will generate linear mixed effects regression models to assess longitudinal changes of above outcomes in response to L. reuteri restoration in human infants, also evaluate the interplay relationship.

Conclusion and significance: This data will confirm that gut L. reuteri being low or absent in HRA infants and restoration of gut L. reuteri can have a long-last effect on improving social function in both high risk infants. This will not only further our understanding of the role of the gut-brain axis in ASD pathogenesis, but also confirm the novel biomarker for ASD early detection, and a promising early intervention that may prevent social deficits in high risk infants later in their life.

Objective: To quantify the presence of gut Limosilactobacillus reuteri (L. reuteri) during early postnatal neurodevelopment can prevent autistic social impairments later in life.

Long term goal: To clinically confirm the early diagnostic biomarkers provided herein and effective early interventions for the high-risk infants for autism spectral disorder (ASD) (HRA, defined below).

Central hypothesis: Multiple heterogeneous upstream molecular and cellular abnormalities caused by genetic and environmental factors lead to L. reuteri-deficiency during perinatal neurodevelopment, which increases the risk of developing autistic social impairments including low responsiveness to human vocal sounds.

Rationale: ASD is a set of complex neurodevelopmental disorders with diverse etiologies that converge to two core disease-defining symptoms 1) social impairments and 2) repetitive behaviors and restricted interests. Despite decades of extensive research, how distinct upstream molecular and cellular abnormalities converge to the ASD core symptoms remains elusive. Also, there are no available medications that can effectively treat these core symptoms. Based on the findings from recent animal studies, we will take a practical approach that bypasses complex and heterogeneous upstream disease mechanisms of individual ASD patients and focuses just on identifying potential final converging points/mechanisms that lead to social impairments. Based on these final converging mechanisms, we hope to develop specific early biomarkers as well as early interventions that can be applied to subgroups of HRA infants to prevent social impairments that otherwise will emerge in toddlerhood.

The central hypothesis will be confirmed with the experiments described in the following Examples.

In Example 1A below, the level of gut L. reuteri in HRA aged 0-6 months will be quantified, and will be compared with that from age matched low risk infants for ASD (LRA), and this level will be correlated with the degree of social impairments and ASD diagnosis at 24 months. Also, quantities of oral supplementation of L. reuteri that can prevent autistic social impairments and ASD diagnosis at 24 months in the HRA infants will be confirmed. It is believed that L. reuteri deficiency is an important converging point of diverse ASD etiologies, as well as a specific biomarker in some HRA infants who are later diagnosed with ASD (HRA-ASD infants). It is also believed that the level of deficiency correlates with the severity of their social impairments.

In Example 1B below, the responsiveness of the HRA and LRA infants to human vocal sounds will be confirmed and this responsiveness of the HRA infants will be correlated with ASD diagnosis at 24 months. It will also be confirmed that oral supplementation of L. reuteri improves the responsiveness of HRA infants to human vocal sounds. It is believed that the lack or reduced responsiveness to human vocal sounds is another specific early biomarker in the HR-ASD infants, which could distinguish them from the LRA infants or from the HRA infants who are not diagnosed with ASD at 24 months (HRA-noASD infants).

### Significance

ASD has become a serious public health crisis due to 1) its rapidly rising prevalence (now 1 in 44 children in the USA¹ and 2) the need of lifelong personal care by caregivers for most patients despite various currently available interventions. Over two thirds of the patients are not able to live independently due to their social and/or intellectual impairments that lead to poor daily living skills²⁻³. The nationwide ASD-related cost is estimated to be around $223 billion in 2Signi020 and is projected to be around $589 billion in 2030⁴. In addition to its enormous negative impact on the public health system, ASD also poses tremendous and long-lasting economic and psychological burdens to the families of patients.

Early diagnosis and early intervention can significantly improve the prognosis of children with ASD. However, an accurate diagnosis is usually made at 2 years of age or older (when behavioral abnormalities emerge) with the Diagnostic and Statistical Manual, Fifth Edition (DSM-5)⁵ or Autism Diagnostic Observation Schedule, Second Edition (ADOS-2)⁶. Therefore, it's urgent to develop early diagnostic tools and effective early interventions for those high-risk children before 2 years of age when there is a better chance, if diagnosed, to correct abnormal brain development trajectory, particularly the development and maturation of key neurotransmitter systems (such as OT system) that are essential to normal social function⁷⁻⁸. However, despite decades of extensive research, the exact mechanisms that underlie ASD pathogenesis remain elusive, and effective treatments, particularly those that can be applied to the children under 2 years of age, are still largely unavailable. Currently, there are no available medications that can effectively treat the core symptoms of ASD.

Examples 1A and 1B here aim to help with both early diagnosis and intervention within the first few months after birth in the HRA infants. These Examples may also shed light on final common pathways of this complex and etiologically diverse disorder that leads to social impairments.

L. reuteri was found to be able to rescue social impairments in multiple mouse models of ASD that have diverse etiologies (environmental, genetic, and idiopathic)⁹⁻¹¹. Our novel and potentially paradigm-shifting hypothesis is that a deficiency of gut L. reuteri could be an important convergent point that leads to social impairments regardless of the upstream disease mechanisms. If our hypothesis is confirmed to be true by this human study with the HRA infants, we not only identify an objective, lab-quantifiable neonatal biomarker for the development of an early diagnosis tool for ASD, but also a very early intervention that can be applied to the HRA infants to potentially prevent social impairments that otherwise will emerge at older ages in some of these infants. This would significantly reduce the ASD-related economic burden on the public health system and remove potential economic and psychological impacts on many families with HRA infants.

Currently, both DSM-5 and ADOS-2, two gold standards usually performed between 2 to 3 years of age for ASD diagnosis, are all based on subjective behavioral evaluations. Typically developing infants, even born prematurely, respond to human voice (particularly their mother's voice) very well¹². If indeed all, or most HRA-ASD infants respond poorly to human vocal sounds and this low responsiveness is corrected at least in some HRA infants with oral supplementation of L. reuteri, the responsiveness to human vocal sounds could be used as another specific early postnatal biomarker that can be objectively measured. Together with the deficiency of gut L. reuteri, these two early postnatal biomarkers can be used to develop a battery of objective tests for ASD diagnosis in early infancy, which would be a major step forward in ASD diagnosis.

### Foundation

These Examples 1A and 1B reflect the following foundational aspects: 1) To identify and verify, in a human study, a potential final common mechanism (gut L. reuteri deficiency) of diverse etiologies (genetic, environmental, and idiopathic) that cause autistic social impairments, and using that mechanism as a substrate/target for both early diagnosis and early intervention in early human infancy. The gut L. reuteri level has not been previously determined in HRA infants, not to mention the role of L. reuteri in ASD pathogenesis in human; 2) To provide building blocks (objective, lab-quantifiable biomarkers) for a battery of specific lab tests that can eventually become an early objective method of ASD diagnosis for high-risk neonatal infants. Currently, ASD diagnosis is based on subjective behavioral evaluations that can only be reliably performed at ages 2 or older. If validated by our study, both gut L. reuteri deficiency and low responsiveness to human vocal sound can be early objective biomarkers with a good predictive value for social impairments that emerges at older ages. 3) To provide an early intervention that could potentially prevent the development of an ASD core symptom (social impairments) at least in some HRA infants. In summary, if the central hypothesis is supported by the results from our human and animal studies, it could be a paradigm shift in both ASD diagnosis and treatment.

### Example 1A—Determine the Level of Gut L. reuteri in HRA Infants (0-6 Months), Compare it With That From Age Matched LRA Infants, and Correlate This Level With the Degree of Social Impairments and ASD Diagnosis at 24 Months; Also Determine Whether Oral Supplementation of L. reuteri Could Prevent Autistic Social Impairments and ASD Diagnosis at 24 Months in HRA Infants

Objective: Determine whether the gut L. reuteri level in early infancy correlates with the degree of social impairments and ASD diagnosis at 24 months of age. Working hypothesis: L. reuteri deficiency could be a final common mechanism of diverse ASD etiologies that leads to social impairments, and therefore could serve as a specific early biomarker at least in some HR-ASD infants. The level of deficiency may correlate with the severity of their social impairments. Approach: Use the real time quantitative polymerase chain reaction (qPCR) to measure gut L. reuteri abundance in all infants (both HRA and LRA infants) 1 day before starting the supplementation as the baseline. Then conduct a randomized controlled trial (RCT) with oral supplementation of L. reuteri for 12 weeks, starting between 2-4 months after birth (due to the technical difficulty in the enrollment and trial management, it is not practical to start the treatment at exactly the same ages as in animal studies). In addition to the baseline level, the level of gut L. reuteri will be determined 4 additional times: 6 and 12 weeks after starting supplementation, and at 12 and 24 months of age. ASD diagnosis is performed at 24 months of age. Correlations between the L. reuteri level at these time points (particularly at the baseline) and ASD diagnosis (especially the sociability sub-scores) at 24 months of age will be analyzed for all groups. Rationale: Recent studies on animal models of ASD with diverse etiologies show a restoration of social function with L. reuteri supplementation administered in juvenile or young adult ASD animals. Based on this, we formed our hypothesis and would like to test it in human HRA infants, translate, and extend (an earlier intervention than that used in the published animal studies) these important findings from the animal research to early diagnosis and treatment of ASD, two highly unmet medical needs in ASD management.

There is growing evidence that gut bacteria can modulate brain signaling and behavior^{11,13-15}. For example, the absence of gut bacteria in germ-free mice causes social impairments in these mice^{10,14}. Oral supplementation of L. reuteri restores sociability in juvenile and young adult animals from etiologically diverse ASD models (environmental¹⁴, genetic⁹⁻¹⁰and idiopathic¹⁰). This effect of L. reuteri is mediated by gut vagal afferent fibers⁰. The vagal activation in turn stimulates release of hypothalamus OT, a neuromodulator in the brain that plays a crucial role in social behavior^{7-8,16}. OT activates dopaminergic neurons in the ventral tegmental area (VTA)^{10-11,14} and neurons in the nucleus accumbens (NAc)^{8,17}, two brain regions critically involved in social cognition and reward. Elevated brain OT signaling in ASD animal models also reorganizes and normalizes connections of the social brain network, which is essential to social rescue in these animals⁸.

Early diagnosis of, and interventions for ASD are more likely to have better long-term outcomes on symptoms and social skills^{3,18}. However, early interventions for the core symptoms of ASD before 2 years of age are very limited, largely because the behavior-based standard diagnosis is performed between 2-3 years of age (more stable diagnosis at 3 years of age)¹⁹, because behavioral abnormalities start to emerge during this age range in the majority of ASD patients. Even after the diagnosis has been made, the interventions are predominantly based on behavioral training and rehabilitation²⁰. Despite available interventions/treatments, more than 80% of patients retain the same level of severity on repeat ADOS assessments over an 8-10-year interval²¹ and most patients have poor daily living skills even at 21 years of age² that do not support their independent living. Indeed, one limited study found that only 12% of adults with ASD and an IQ of at least 70 lived independently²². These clinical results indicate low effectiveness of the available interventions/treatments and an urgent need for more effective interventions, particularly those that can be applied before 2 years of age.

For any early interventions/treatments with medications (including natural products) before 2 years of age, a major concern is their safety—potential long-term adverse side effects. L. reuteri is a symbiotic probiotic that can be found in mother's milk²³ and is widely present in the gut of all mammals, including human²⁴⁻²⁶ and wild-type mice¹⁴. Furthermore, it has been used in several clinical studies involving infants²⁷⁻³⁰ with a good safety profile²⁹⁻³⁰. Thus, it is safe to be used as an early intervention for the HRA infants if it is effective.

### Summary of Preliminary Data and Findings

L. reuteri supplementation in children with Prader-Willi syndrome (PWS) improves social function. Our group has conducted and published a human RCT with L. reuteri mediated intervention in children with PWS³¹. To our knowledge, this is the first and only RCT that used L. reuteri for the treatment of social impairments. PWS is a rare genetic disorder associated with developmental delay, obesity, and neuropsychiatric comorbidities including social impairments³². It is considered as a type of syndromic ASD, as the prevalence of ASD among PWS patients is significantly higher than that of general population³³. After a 12-week treatment with L. reuteri oral supplementation, we found a significant improvement in social function in patients with PWS³¹. The improvement was evaluated with Gilliam Autism Rating Scale-3rd edition (GARS-3), a popular norm-referenced tool designed to screen for ASD in individuals between ages of 3 and 22³⁴. In the Social Communication subscale, the sub-scores (higher scores indicate more severe behavioral concerns) of the placebo and treated groups are 18.8±5.93 and 11.5±6.01, respectively (p=0.007); In the Social Interaction subscale, the sub-scores of the placebo and treated groups are 13.1±8.55 and 7.69±5.57, respectively (p=0.037)³¹.

Evidence for L. reuteri deficiency in ASD patients. In a retrograde review of Dr. Kong's preliminary and informal clinical observation, it was found that the abundance of gut L. reuteri in all her young ASD patients (n=14) was undetectable, while the test lab reference median value was 0.002 (Table below). Since it was not a formal study, Dr. Kong did not include an age-matched control group. Two studies are intended to investigate this issue in more details: one for determining the abundance of gut L. reuteri from both ASD and age matched typically developing children, and another for determining the abundance of gut L. reuteri in 0-6-month high risk and age matched low risk infants for developing into ASD. Meanwhile, we would like to point out that our clinical observation data shown in Table below is consistent with what is observed in a human data base https://gmrepo.humangut.info/phenotypes/D000067877, which (using a metagenomic assay) also shows that gut L. reuteri, as a species, is undetectable, but the Lactobacillus genus from ASD children is either high (6 out of 14 ASD children) or low (8 out of 14 ASD children). This variability in the level of Lactobacillus genus is consistent with the results reported³⁵⁻³⁷, independent of gut L. reuteri level as a species.

TABLE 1
Genus- and species-level Lactobacilli and L. reuteri relative abundances
determined by metagenomic measurement (WMS) from Guhe Health.

| | | | | Reference | | Reference |
|---|---|---|---|---|---|---|
| | | ASD | Lactobacillus | value | L. reuteri | value |
| Age | Sex | severity | (% RA) | (median) | (% RA) | (median) |
| 3 y 5 m | male | severe | 0.003 | 0.01 | 0 | 0.002 |
| 5 y 11 m | female | moderate | 4.753 | 0.01 | 0 | 0.002 |
| 4 y 5 m | male | moderate | 0 | 0.01 | 0 | 0.002 |
| 6 y 6 m | male | mild | 0 | 0.01 | 0 | 0.002 |
| 6 y 4 m | male | moderate | 0 | 0.01 | 0 | 0.002 |
| 1 y 8 m | male | mild | 0 | 0.01 | 0 | 0.002 |
| 5 y | male | mild | 0.83 | 0.01 | 0 | 0.002 |
| 3 y 6 m | female | mild | 1.833 | 0.01 | 0 | 0.002 |
| 2 y 5 m | male | severe | 0 | 0.01 | 0 | 0.002 |
| 6 y 10 m | male | severe | 9.084 | 0.1 | 0 | 0.002 |
| 2 y 3 m | male | severe | 3.52 | 0.1 | 0 | 0.002 |
| 3 y | male | moderate | 2.627 | 0.01 | 0 | 0.002 |
| 4 y | male | moderate | 0.003 | 0.01 | 0 | 0.002 |
| 5 y | male | moderate | 0 | 0.01 | 0 | 0.002 |

The undetectable levels of L. reuteri in Table 1 above and from the data base mentioned above were all determined with metagenomic measurement (WMS). In our newly designed ongoing studies, we will use a more sensitive and specific method (qPCR) to measure gut L. reuteri levels. This would allow us to detect lower levels of gut L. reuteri in both ASD children and HRA infants and compared them with those from age matched controls. We expect to see a difference in the abundance of gut L. reuteri between these high vs low risk infant groups, similar to what has been reported between Cntnap2 KO and wild-type (WT) mice when qPCR was used to determine the abundance of gut L. reuteri¹¹. We will compare the results obtained with qPCR and metagenomics. We will also determine the level of gut tetrahydrobiopterin (BH4), a metabolite of L. reuteri that was able to rescue social impairments in Cntnap2 KO mice¹¹. The methodologies used to study the gut microbiome, including sample collection/handling and data analysis/interpretation, are detailed in previous publications³⁸⁻⁴⁵.

### Study Population and Procedure

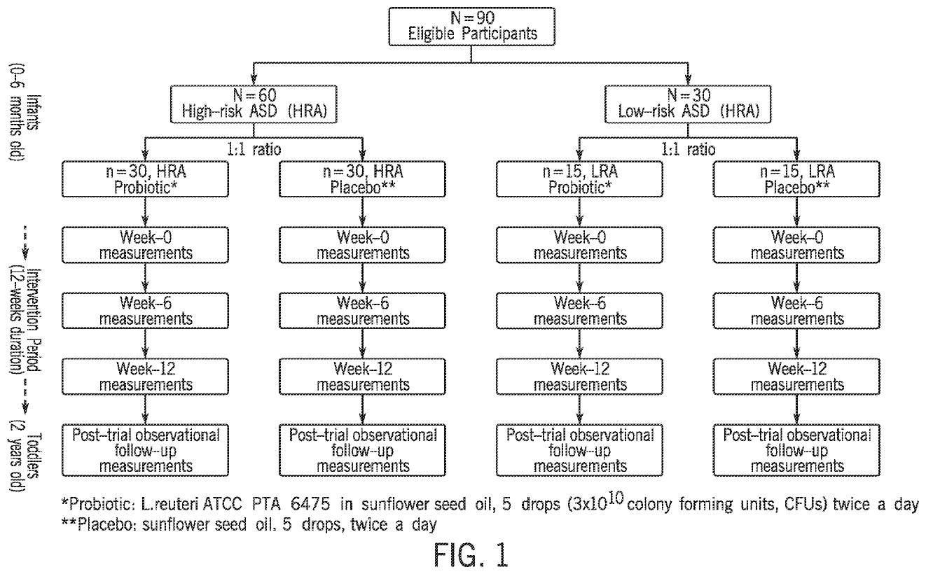

The study population will be infants aged 0-6 months with either high risk (HRA) or low risk (LRA) for ASD. The HRA infants are defined as those who have a sibling diagnosed with ASD, or children whose parents and caregivers suspect developmental delays and have an Ages and Stages Questionnaire-Third Edition (ASQ-3)⁴⁶ score “Monitor” and/or “Fail” in the Communication domain. Here we use ASQ-3 as one of the inclusion criteria because the previous study⁴⁷ screened 2,848 toddlers with the ASQ-3 across 20 pediatric sites, used the “monitor and/or fail” cutoff on any domain, the ASQ-3 identified 83% of those later diagnosed with an ASD. Another study tested 124 diagnosed with ASD, ASQ-3 identified 82% by the Communication domain⁴⁸. The LRA infants are defined as no family history of ASD or full term without developmental concerns and ASQ-3 score with “pass”. A phone screen will be performed to assess subjects' eligibility. We plan to include 60 HRA infants and 30 LRA infants, randomly and double blinded assign into L. reuteri or placebo groups for 12 weeks then follow up these infants when they reach 24 months. The L. reuteri stain (ATCC PTA 6475) can restore OT signaling in virtually all the animal studies cited above was originally isolated from the milk of a healthy breastfeeding human mother²³. The study flowchart is as showed (FIG. 1). We will collect stool samples for qPCR of L. reuteri (the primary exposure of interest) and Metagenomics and BH4 along with a number of psychometric tests: ASQ-3, Mullen Scale of Early Learning (MSEL)⁴⁹⁻⁵⁰, Vineland Adaptive Behavioral Scale Third Edition (Vineland-3)⁵¹ at week 0, 6, 12 of intervention during the RCT, and at age 12 and 24 months, DSM-5 and ADOS-2 will be performed at 24 months of age, the time schedule is as showed below Table 2 below.

TABLE 2
The schedule of evaluations during the study and follow-up

| | | RCT | RCT | | |
|---|---|---|---|---|---|
| | RCT | 6 | 12 | 12 | 24 |
| Measurement | Baseline | weeks | weeks | months | months |
| qPCR for | X | X | X | X | X |
| gut L. reuteri | | | | | |
| Metagenomics | X | | X | | X |
| ELISA for BH4 | X | | X | | X |
| ASQ-3, MSEL, | X | X | X | X | X |
| Vineland-3 | | | | | |
| ADOS-2, DSM-5 | | | | | X |

### Methodology

### qPCR FOR SPECIES-LEVEL QUANTIFICATION OF STOOL L. REUTERI:

Stool samples will be collected at five study timepoints: prior to intervention 0-weeks, after intervention 6-weeks and 12-weeks, post RCT when these infants reached 12 months and 24 months. Sample collection will be performed with OMNIgene Gut OMR-200 and OMNImet Gut ME-200 collection kits (DNA Genotek Inc.) by the participants at home under the supervision of trained parents and stored at room temperature, before de-identification and pick up or delivery to the Athinoula A. Martinos Center, where stool samples will be stored at −80° C. freezer. The level of L. reuteri in stool will be determined using qPCR with L. reuteri species-specific PCR primer pairs (LR-F: 5′-GATTGACGATGGATCACCAG-3′ (SEQ ID NO: 1); LR-R: 5′-ACTACCAGGGTATCTAATCC-3′ (SEQ ID NO: 2)) and the respective TaqMan probes, EXPRESS SYBR GreenER (Life Technologies), and a CFX96 Real-Time System. The amplification program consists of 1 cycle of 95° C. for 3 min, followed by 40 cycles of 95° C. for 40 s, 56° C. for 30 s, and 72° C. for 1 min; finally, extension is carried out at 72° C. for 8 min. L. reuteri will then be quantified based on common methods. That is, standard DNA will be extracted from a dried powder of a known concentration of L. reuteri in CFUs per gram. The extraction method is the same as that used for unknown samples. Standard DNA diluted standard DNA, and unknown sample DNA will be simultaneously analyzed by qPCR. The standard curve for quantification will be constructed using the CT values of the standard DNA and the diluted standard DNA samples. Quantification of L. reuteri in the unknown sample will be calculated based upon comparison of the corresponding CT values with the standard curve¹¹.

STOOL METAGENOMIC AND BH4 IMMUNOASSAY: The collected stool samples will be hand delivered with dry ice packaging to Massachusetts Host-Microbiome Center+Crimson Core at Brigham & Woman's Hospital for DNA extraction and Metagenomics sequencing analysis to check both microbiome composition (including L. reuteri abundance) & functional profiles. Microbial DNA will then be extracted according to the manufacturer's instructions, and DNA samples will be quantified with a NanoDrop spectrophotometer. A260/A280 ratios will be also measured to confirm high-purity DNA yield. Stool BH4¹¹ will be measured by ELISA via Arbor assay.

One example stool metagenomic sequencing analysis is described in reference 41 (Kong et al., Front Nutr. 2021 Feb. 19;8:587974. doi: 10.3389/fnut.2021.587974. PMID: 33681271; PMCID: PMC7933553) hereby incorporated by reference in its entirety. In summary, DNA from the stool samples will be subjected to 16S rRNA Gene Amplicon Sequencing. The 16S rRNA V3-V4 library will be constructed by two rounds of PCR with the following primers: 341F: 5′TCGTCGGCAGCGTCAGATGTGTATAAGAGACAGCCTACGGGAGGCAGCAGCC TACGGGNBGCASCAG3′ ID NO: (SEQ 3) and 805R:5′GTCTCGTGGGCTCGGAGATGTGTATAAGAGACAGTGACTACNVGGGTATCTA ATCC3′ (SEQ ID NO: 4) via reaction procedure (95° C. for 2 min, followed by 25 cycles at 95° C. for 30 s, 55° C. for 30 s, and 72° C. for 30 s, and a final extension at 72° C. for 5 min) PCR products will be purified with 1× KAPA AMPure beads (KAPA, Cat #KK8002). Then, products will be put through a second PCR reaction procedure (95° C. for 2 min, followed by eight cycles at 95° C. for 30 s, 55° C. for 30 s, and 72° C. for 30 s, and a final extension at 72° C. for 5 min). PCR products will be purified with 1× KAPA AMPure beads and will be analyzed using a Bioanalyzer DNA kit, followed by quantification with real-time PCR. DNA libraries will be pooled and sequenced on Illumina MiSeq (Illumina; CA) using a 2×250 bp paired-end protocol with overlapping reads.

PSYCHOMETRIC AND BEHAVIORAL TESTS: ASQ-3; MSEL. Vineland-3 will be performed at week 0, 6, 12 of the trial and when they reached 24 months to check social function. ADOS-2 and DSM-5 will be done at age 24 months.

STATISTICS: All raw clinical data will be recorded and processed in Microsoft Excel 2007 and R 4.1.2 (or later). The presentation of data follows the CONSORT recommendations for reporting results of RCTs. Descriptive summaries, both tabular and graphical, will be generated for all covariates by risk group (low, high), treatment (placebo, probiotic), and time (0, 6 weeks, 12 weeks, 24 months). Continuous variables will be summarized as mean (standard deviation) and median (interquartile range), while categorical variables will be summarized as frequencies and percentages. Boxplots and scatterplots will be generated to visually summarize distributions and pairwise associations between all variables of interest. Pairwise associations will also be quantified using Spearman correlation coefficients. Group comparisons will be performed using either the chi-square test or Fisher's exact test or the analysis of variance or Mann-Whitney test, respectively.

A generalized linear mixed-effects model will be constructed to quantify the association between the level of gut L. reuteri [copies/ng] and risk group, treatment, and time. Given theoretical range of the level of L. reuteri (>0), we will evaluate different link functions and distributional families to identify the family that is best supported by the data. For example, we plan to explore the following link/family combinations: linear (identity, normal) and proportional odds (cumulative logit, binomial). All fixed-effects variables will be modeled as indicators, and we plan to explore the need to two-and three-way interactions. Additional adjustment variables, such as age and sex, will be included in all secondary models. Non-linear relationships between each outcome each will be explored by comparing model fits when using restricted cubic spline and linear coding of continuous variables. Random effects will be incorporated into the specification of the conditional mean model using either intercepts or lines (intercepts and slopes) to account for multiple measurements per participant. Linear combinations of fixed-effects, along with their 95% confidence intervals, will be computed to summarize key effects including cross-sectional treatment and risk group comparisons (e.g., probiotic vs placebo at baseline, high risk vs low risk at baseline) and temporal comparisons (e.g., week 6 vs week 0 amongst high-risk participants at baseline). Due to having several ancillary outcomes and both temporal and cross-sectional comparisons, false discovery rate (FDR)-adjusted P-values will be used to account for multiple comparisons. All attempts will be made to minimize missing data, but we will perform the previously described analyses using only those with available data (complete-case analysis) as well as performing multiple imputation under the missing at random assumption. If needed, we will construct at least 50 imputed data sets. The above analyses will be performed on each imputed data set, and the resultant vector of parameter estimates, and covariance matrices will be combined using Rubin's rules.

Demultiplexed, paired-end reads from whole metagenome shotgun sequencing (WMS) will be processed through read-level quality control, taxonomic profiling, functional profiling, and strain profiling via BioBakery Workflows and included software packages⁵² with default parameters for the whole metagenome shotgun workflow to generate merged, count feature tables for downstream statistical analysis. Subsequent statistical analysis of processed WMS data will be performed via R. α- and β-diversity metrics will be computed and visualized via the Phyloseq package under R. Differences in abundance of bacterial species and pathways both between groups and between study visits will be determined using Wilcoxon rank sum test with statistical significance considered at p<0.05. The linear discriminant analysis (LDA) effect size (LEfSe) algorithm⁵³ will be used to identify specific bacterial taxa and metabolic pathways as taxonomic and functional biomarkers and to verify the Wilcoxon rank sum test. Statistical significance is considered at p<0.05 for class-wise comparisons via Kruskal-Wallis tests following the LEfSe pipeline. Additionally, a discriminative feature is considered using a threshold for the logarithmic LDA score set at >2.

### Expected Outcomes From the Above Experiments

It is expected that 1) HRA-ASD infants have an L. reuteri deficiency in the gut, whereas the HRA-noASD infants will have either a similar level or somewhat reduced level of gut L. reuteri compared with that of LRA infants; 2) the level of gut L. reuteri correlates to the severity of social impairments and an ASD diagnosis in HR-ASD infants; and 3) oral supplementation of L. reuteri reduces the chance or even prevent the development into ASD at 2 years of age.

### Potential Pitfalls and Alternative Strategies

Potential pitfalls are: 1) the gut level of L. reuteri in some HRA-ASD infants may not be reduced compared with those of HRA-noASD and LRA infants at all 5 time points (see approach above). According to recent animal studies, L. reuteri mediated activation of the vagal afferent stimulates synthesis and release of hypothalamus OT¹⁰ that in turn activates multiple down-stream targets in the brain and connects the social brain network that is crucial to normal social function⁸. Mechanistically, even there is NO L. reuteri deficiency in the gut, any disruptions along the brain OT pathway (downstream of L. reuteri action and vagal stimulation of OT synthesis) could still lead to social impairments. For example, lost function mutations in genes involved in OT release⁵⁴, or transmission⁵⁵ contribute to pathogenesis of ASD in human patients. Therefore, an alternative strategy is to do a genetic test to check the highly penetrant disease causal mutations of the genes involved in the OT pathway for these HRA infants with no L. reuteri deficiency. For some of these HRA infants, if their genetic defects cause the disruption of OT expression and release, their social impairments that emerge later in life could be ameliorated with administration of exogenous OT. 2) a deficiency of gut L. reuteri is observed in a subgroup of HRA-ASD infants, but no improvement of social impairments with oral L. reuteri supplementation at 2 years of age. An alternative strategy is again to do a genetic test for these infants to identify the highly penetrant disease causal mutations of the genes associated with ASD. Lost function mutations in the OT receptor gene⁵⁵, or in genes involved in enabling OT signaling in some key social brain areas, such as ventral tegmental area⁵⁶, may explain our results. In this case, administration of exogenous OT will not improve social impairments. These unexpected results and findings of the gene test are still valuable, as they can guide the patient selection process based on their individual mutation profiles to include or exclude patients for clinical trials with exogenous OT. They could also help to explain why some clinical trials using OT nasal spray for the treatment of ASD have failed.

### Example 1B: Determine the Responsiveness of the HRA and LRA Infants to Human Vocal Sounds and Correlate This Responsiveness of the HRA Infants With ASD Diagnosis at 24 Months; and Determine Whether Oral Supplementation of L. reuteri Improves the Responsiveness of the HRA Infants to Human Vocal Sounds and Reduces the Number of These Infants Developing into ASD at 24 Months

Objective: Determine whether lack or reduced responsiveness to human vocal sounds can be an early specific biomarker for ASD. Also, determine whether the responsiveness can be an independent endpoint for the efficacy of neonatal supplementation of L. reuteri. Working hypothesis: Lack or reduced responsiveness to human vocal sounds is likely another specific early biomarker in those HRA-ASD infants, which could distinguish them from the LRA infants or from the HRA infants who are not diagnosed with ASD at 24 months (HRA-noASD infants). This deficit can be restored with neonatal supplementation of L. reuteri. Approach: The responsiveness to human vocal sounds in all infants will be evaluated by 1) recording event-related potentials (ERP, time-locked EEG signals) that are induced by human vocal sounds and non-vocal sounds; 2) measuring the OT level in saliva with an immunoassay before and after exposure to human vocal sounds (mother's and other human voice) and non-vocal sounds (telephone rings or other environmental sounds). The degree of the responsiveness to human vocal sounds will be correlated with the severity of social impairments and ASD diagnosis at 24 months in HRA infants with and without oral supplementation of L. reuteri in a RCT. Rationale: Low responsiveness to human vocal sounds in infancy could be an independent early biomarker of ASD. It could also be used as a biomarker for the efficacy of early interventions. We will apply both electrophysiological and biochemical approach to objectively evaluate the responsiveness of HRA infants to human vocal sounds and correlate it to the degree of social impairments and ASD diagnosis at 2 years of age. If the results from two types of experiment agree with each other and correlate well with ASD diagnosis, the low responsiveness to human vocal sounds can be used as a good tool to help with early detection of ASD as well as evaluation of the efficacy of an early intervention for ASD.

For typically developing infants, the human vocal sound, particularly the mother's voice, appears to be an effective stimulus conducive of social engagement and bonding, such as mother-infant bonding⁵⁷⁻⁵⁸. Even in preterm infants who need to be in the newborn intensive care unit after birth for medical reasons, mother's voice can alleviate pain during painful procedures, indicating these preterm infants can hear and respond to mother's voice effectivelyl¹² Furthermore, newborns can discriminate mother's voice over unfamiliar female's voice58-60 and even detect fine differences in speech-like sounds⁶¹⁻⁶². This early responsiveness to human vocal sounds and competency of discriminating and extracting subtle details of vocal sounds probably subserve a variety of developmental functions such as language acquisition^{58,61}. In older children (7-12 years), mother's voice delivered even remotely from telephone can calm down stressed children, relief their anxiety, and provide social support through stimulating OT release in these children⁶³.

On the other hand, it has long been recognized since the classic description of the disorder that young autistic children are often oblivious to the human vocal sounds⁶⁴⁻⁶⁷. This abnormal auditory perception seems already exist in early infancy of HRA infants later diagnosed with ASD⁶⁸. The selectively reduced responsiveness to human vocal sounds (particularly to parents' voice) is sometimes the first sign noticed by parents, often leading to a referral for audiological examination⁶⁶. It has been shown with brain imaging of adults with ASD that the superior temporal sulcus voice-selective regions were not activated in response to human vocal sounds, but a normal activation pattern was observed in response to non-vocal sounds⁶⁹, indicating selectively impaired cortical processing of human vocal sounds in ASD patients. Similar conclusion has been made from an imaging study on the brain network that is involved in processing human vocal sounds in children with ASD⁶⁷. Abnormal processing of human vocal sounds has also been demonstrated at subcortical levels in the brainstem⁷⁰⁻⁷¹. Thus, lack of, or reduced response to human vocal sounds can be detected in HRA-ASD infants (those diagnosed with ASD later in toddlerhood) and in children and adults with ASD.

Low responsiveness to human vocal sounds/mother's voice appears to be specific to autistic children, since age-matched intellectual disabled children, similar to typically developed children, show strong preference for their mothers' speech over environmental sounds (the noise of superimposed unfamiliar voices)⁶⁶. The responsiveness to human vocal sounds/mother's voice can be evaluated noninvasively in infants (including preterm infants) with both the ERP^{59,72-73}, and measurement of saliva OT level¹², a method that is also been used in juveniles⁶³.

### Summary of Preliminary Data and Findings

Alterations in human vocal/speech evoked brain response and in brain structures in children with ASD. We recently published a systemic review⁷⁴ to compare early screening value of measuring cortical response via EEG (41 studies), fNIRS (8 studies) and MRI (26 studies) in infancy and concluded that the inability to recognize human vocals and lower responsiveness to social stimuli in high-risk infants correlated with their social impairment and ASD diagnosis in toddlerhood. We also studied abnormal brain processing and structures with EEG/ERP^{70,75} fMRI⁷⁶⁻⁷⁸ and fNIRS⁷⁹. More specifically, we assessed the developmental pattern of auditory information processing of human speech at the level of the brainstem in preschool children with or without ASD using speech-evoked auditory brainstem response (speech-ABR) only⁷⁰ or with structural magnetic resonance imaging (sMRI)⁷⁵. Our results show that subcortical auditory processing of human speech is impaired and underdeveloped in children with ASD compared with age-matched, typically developing children. Thus, the expertise and experience in using noninvasive electrophysiological and brain imaging tools to study abnormal processing of human vocal sounds at subcortical and cortical levels exists, which will aid in accomplishing the experiments of Example 1B.

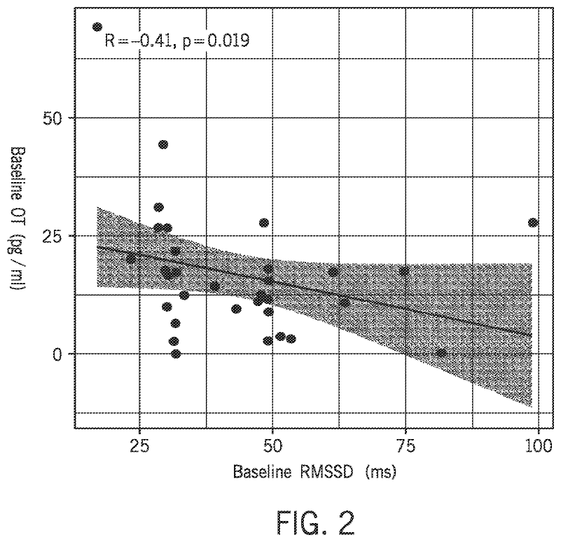

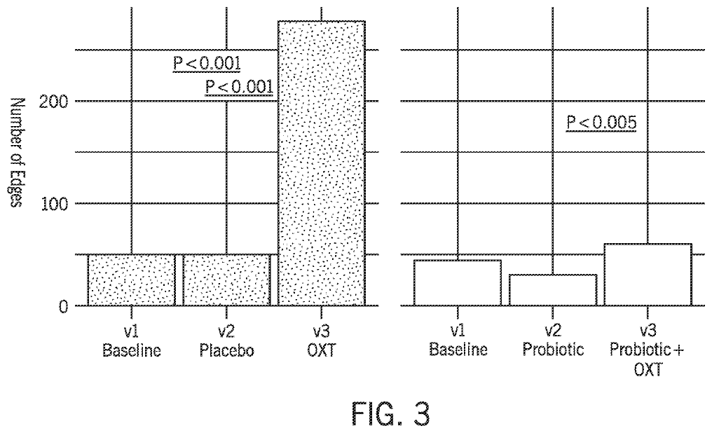

Oxytocin (OT), gut dysbiosis, autonomic nervous system (ANS) function in ASD and ASD subgrouping. In a recent study, we examined the correlation between serum OT level and dysbiosis of gut microbiome in children with ASD and compared it with the age-matched normal children⁸⁰. We found that the serum OT level of ASD children was lower compared with that of the healthy control group (P<0.05) and this reduced OT level was correlated to the degree of gut dysbiosis⁸⁰. In addition, we found that baseline level of OT correlated with the baseline root mean square of successive differences between normal heartbeats (RMSSD) (FIG. 2) which is reported highly relevant ANS index to ASD⁴⁰. In another study, we tried to distinguish children with ASD from their sibling controls based on ANS index, combined ANS and microbiome markers that correlated with ASD core symptoms and severity⁴⁰. We also found in a previously RCT that OT nasal application for 3 months significantly increased microbiome co-occurrence networks³⁸ (FIG. 3, OXT=OT). Additionally, we tried to expand and improve the utility of RITA-T (a rapid and inexpensive behavioural ASD screen tool for toddlers (18-36 months) into a broader age range (18-84 months) and validate the accuracy of the screen results with ADOS-2⁸¹. We also made an effort to improve the eye tracking paradigm so that the testing with young ASD children can be performed more smoothly, which improves the quality of the test results⁸²⁻⁸³ Developing ANS and gut microbiome markers for ASD and ASD subgrouping are two other areas where significant progresses have been made in these areas^{39-40,42-43}. In summary, the above-mentioned recent work has established a solid foundation in the methodology as well as a conceptual framework for carrying out the experiments necessary for success with Example 1B.

The study population and procedure for these experiments in Example 1B, study flowchart is the same as described above in Example 1A—see FIG. 1. Instead of collecting stool samples, we will collect saliva sample for OT and cortisol and blood samples for OT level and cytokines at week 0, 6, 12 of RCT, and when they reached 24 months, meantime check EEG/ERP response to human vocals (the primary exposure of interest) and other social stimuli, autonomic parameters via wrist band and pupil size via eye tracking to human vocals and social stimuli, psychometric tests for social function and sensory profile (defined under methodology below). The time schedule is as showed below Table 3 below.

TABLE 3
Study instruments by timepoint

| | | RCT | RCT | |
|---|---|---|---|---|
| | RCT | 6 | 12 | 24 |
| Measurement | Baseline | weeks | weeks | months |
| EEG | X | X | X | X |
| Saliva OT/Cortisol | X | X | X | X |
| Blood OT/cytokines | X | | X | X |
| Wristband (Autonomic index) | X | X | X | X |
| Eye tracking (Pupil size) | X | X | X | X |
| ASD-3, MSEL, Vineland-3, | X | X | X | X |
| ITSP | | | | |
| ADOS-2, DSM-5 | | | | X |

### Methodology

We believe that low or lack of responsiveness to human vocal sounds could serve as an early and specific biomarker with predictive value for ASD, as well as a potential clinical endpoint to evaluate the efficacy of oral supplementation of L. reuteri in HRA infants.

To experimentally evaluate the responsiveness to human vocal sounds in all 3 groups of infants, we will use two technically different, but validated approaches: 1) determine mother's voice-induced elevation of saliva oxytocin, and 2) record ERP elicited by mother's voice. Mother's voice is a strong social signal that stimulate release of oxytocin in children, ranging from preterm infants¹² to juveniles⁶³.

EEG/ERP RESPONSIVENESS TO HUMAN VOCALS: EEG (Brain Vision) will be used to study electrophysiological responses to auditory and visual stimuli in subjects. ERP will be compared across different conditions recorded from 60 scalp sites to test in an ERP paradigm^{72,84}. The amplitude and latency of components (P100, P200, MMN, N400) will be provided as the main outcome of ERPs. In addition, we will check spectral content such as mu wave activity and oscillatory synchrony between different brain regions, especially brain areas involved in social communication. The baseline EEG will be recorded for 2 min before the auditory stimuli is presented through two loudspeakers placed 65 cm away from each side of infant's head⁵⁹. Auditory stimuli are comprised by a set of pre-recorded audios of mother (10%) or unfamiliar female talking (20%) and environmental sounds 70% which are digitalized and edited to 750 ms. Total 200 trials will be presented with interstimulus interval (ISI) about 2500 ms. The stimulus intensity is about 65 dB, and background noise should be less than 10 dB^{12,85-86} acquired via a calibrated sound level meter (Volt-craft Phonometer SL-10; Conrad Electronic, Hirschau, Germany). Visual stimuli present a still and light scenery picture during the first 100 trials of auditory stimuli, then present alternative human faces and toys with full colored life size, woman talking/telling nursery rhythm and dynamic toys such as a ball dropping down a chute each 10 seconds⁸⁷⁻⁸⁸. Video sets will be presented in random order and repeated twice (e.g., SNSN). Stimuli will be presented on a 23-inch monitor using E-Prime with the infant seated 65 cm from the monitor^{68,89}. The duration of this paradigm is about 12 min.

AUTONOMIC RESPONSE TO HUMAN VOCALS: Simultaneously, the infants will wear Empatica E4 wristbands for data collection to record Heart rate variability (HRV), blood volume pulse (BVP), peripheral skin temperature, electrodermal activity (EDA), and 3-axis accelerometer data for motion detection^{40,90}, the response to human vocals^{12,91}, the pupil size diameter and the change with social stimuli will be measured via an eye-tracker (Tobii Pro)⁹² data sampled at 250Hz.

SALIVA AND SERUM OT IMMUNOASSAY: Saliva samples^{12,93} using SalivaBio Infant Swab and blood samples will be collected at the scheduled time illustrated on table 3, immediately store them at −80 degree after collection, will test OT level with human ELISA kits from Arbor Assays. Additional saliva and blood will be used for checking cortisol and cytokines via ELISA kits of Arbor Assays.

PSYCHOMETRIC AND BEHAVIORAL TESTS: The Infant/Toddler Sensory Profile (ITSP)⁹⁴ along with ASQ-3, MSEL, Vineland-3 at 0,6,12 weeks and 24 months, ADOS-2 and DSM-5 at 24 months.

STATISTICS: Pre-processing and spectral/time-series and ERP analysis of raw EEG data will be performed using BESA Research 7.1 and BESA Statistics 2.1. Derived EEG features from the spectral/time-series and ERP analysis, such as μ wave spectral power, will be statistically analyzed in R. Collected photoplethysmograph (PPG) sensor data from the Empatica E4 wristband will be pre-processed by epoch filtering using built-in noise detection algorithm from the Kubios HRV software. Furthermore, HR- and HRV-associated parameters will be derived from time-domain analysis of each participant 12-minute data collection interval. A similar modeling approach, to that of Example 1A, will be adopted when separately quantifying the association between other outcomes (e.g., social impairments, responsiveness (EEG, eye-ball tracking) and lagged L. reuteri concentration, lagged time, risk group, and treatment group. For outcomes only collected at 24 months (e.g., ASD diagnosis), only fixed-effects generalized linear models will be considered along with aggregated summaries of L. reuteri concentration (e.g., baseline, slope, range).

### Expected Outcomes from the Experiments of Examle 1B

It is expected that 1) both electrophysiological and biochemical methods identify untreated (without oral supplementation of L. reuteri) HRA-ASD infants before they are diagnosed at 2years of age; 2) the responsiveness to human vocal sounds correlate to the severity of social impairments and an ASD diagnosis in HRA-ASD infants; and 3) oral supplementation of L. reuteri enhances the responsiveness and prevent the development into ASD at 2 years of age.

### Potential Pitfalls and Alternative Strategies

A potential pitfall is that the low responsiveness to human vocal sounds in some HRA-ASD infants is not improved with oral L. reuteri supplementation. According to recent animal studies, L. reuteri mediated activation of the vagal afferent stimulates synthesis and release of hypothalamus OT¹⁰. This in turn activates multiple down-stream targets in the brain and connects the social brain network that is crucial to normal social function⁸. Lack or low responsiveness to human vocal sounds in HRA-ASD infants could be caused by weakened OT system⁹⁵. If this is true, any disruptions along the brain OT pathway (downstream of L. reuteri action and vagal stimulation of OT synthesis) could still lead to low responsiveness to human vocal sounds. For example, lost function mutations in genes involved in OT release⁹⁶, facilitation of OT receptor expression⁹⁷, or encoding OT receptor⁵⁵ can all lead to pathogenesis of ASD in human patients. In those cases, oral L. reuteri supplementation may have no effect. Therefore, an alternative strategy is to do a genetic test to check the known highly penetrant disease causal mutations of the genes involved in the OT signaling pathway for these HRA infants with no L. reuteri deficiency. For some of these HRA infants, if their genetic defects are limited only to the disruption of OT expression and release, their social impairments that emerge in toddlerhood could be ameliorated with administration of exogenous OT⁹⁸⁻⁹⁹.

### Summary

The experiments in Examples 1A and 1B aim to 1) identify objective (lab-quantifiable) early postnatal diagnostic biomarkers and 2) develop an effective early intervention for those infants who are at high risk to develop into ASD later in life. We believe that the lack of specific L. reuteri—elicited vagal input from its gut branches impairs the early development and maturation of the hypothalamus OT system. This in turn causes reduced oxytocinergic signaling to various social brain regions, which eventually leads to autistic social impairments. If this is supported by the results from our buman and animal studies described herein, this not only identifies gut L. reuteri deficiency as a final common mechanism in human ASD, regardless of diverse and complex upstream disease mechanisms of individual patients but could be a paradigm shift in both ASD diagnosis and treatment as well.

### REFERENCES

1. Maenner MJ, Shaw KA, Bakian AV, Bilder DA, Durkin MS, Esler A, Furnier SM, Hallas L, Hall-Lande J, Hudson A, Hughes MM, Patrick M, Pierce K, Poynter JN, Salinas A, Shenouda J, Vehorn A, Warren Z, Constantino JN, DiRienzo M, Fitzgerald RT, Grzybowski A, Spivey MH, Pettygrove S, Zahorodny W, Ali A, Andrews JG, Baroud T, Gutierrez J, Hewitt A, Lee LC, Lopez M, Mancilla KC, McArthur D, Schwenk YD, Washington A, Williams S, Cogswell ME. Prevalence and Characteristics of Autism Spectrum Disorder Among Children Aged 8 Years—Autism and Developmental Disabilities Monitoring Network, 11 Sites, United States, 2018. MMWR Surveill Summ. 2021 Dec. 3;70(11):1-16. doi: 10.15585/mmwr.ss7011a1. PMID: 34855725; PMCID: PMC8639024.
2. Bal VH, Kim SH, Cheong D, Lord C. Daily living skills in individuals with autism spectrum disorder from 2 to 21 years of age. Autism. 2015 October; 19(7):774-84. doi: 10.1177/1362361315575840. Epub 2015 Apr. 28. PMID: 25922445; PMCID: PMC4912002.
3. Sanchack KE, Thomas CA. Autism Spectrum Disorder: Primary Care Principles. Am Fam Physician. 2016 Dec. 15;94(12):972-979. PMID: 28075089.
4. Blaxill M, Rogers T, Nevison C. Autism Tsunami: the Impact of Rising Prevalence on the Societal Cost of Autism in the United States. J Autism Dev Disord. 2022 June; 52(6):2627-2643. doi: 10.1007/s10803-021-05120-7. Epub 2021 Jul. 18. PMID: 34278527; PMCID: PMC9114071.
5. American Psychiatric Association. Diagnostic and Statistical Manual of Mental Disorders: DSM-5. 5th ed. Washington, DC: American Psychiatric Association (2013).
6. Lord C, Rutter M, DiLavore PC, Risi S, Gotham K, Bishop S. (ADOSTM-2) Autism Diagnostic Observation ScheduleTM, Second Edition. WPS (2012).
7. Peñagarikano O, Lázaro MT, Lu XH, Gordon A, Dong H, Lam HA, Peles E, Maidment NT, Murphy NP, Yang XW, Golshani P, Geschwind DH. Exogenous and evoked oxytocin restores social behavior in the Cntnap2 mouse model of autism. Sci Transl Med. 2015 Jan. 21;7(271):271ra8. doi: 10.1126/scitranslmed.3010257. PMID: 25609168; PMCID: PMC4498455.
8. Choe KY, Bethlehem RAI, Safrin M, Dong H, Salman E, Li Y, Grinevich V, Golshani P, DeNardo LA, Peñagarikano O, Harris NG, Geschwind DH. Oxytocin normalizes altered circuit connectivity for social rescue of the Cntnap2 knockout mouse. Neuron. 2022 Mar. 2;110(5):795-808. e6. doi: 10.1016/j.neuron.2021.11.031. Epub 2021 Dec. 20. PMID: 34932941; PMCID: PMC8944915.
9. Tabouy L, Getselter D, Ziv O, Karpuj M, Tabouy T, Lukic I, Maayouf R, Werbner N, Ben-Amram H, Nuriel-Ohayon M, Koren O, Elliott E. Dysbiosis of microbiome and probiotic treatment in a genetic model of autism spectrum disorders. Brain Behav Immun. 2018 October; 73:310-319. doi: 10.1016/j.bbi.2018.05.015. Epub 2018 May 19. PMID: 29787855.
10. Sgritta M, Dooling SW, Buffington SA, Momin EN, Francis MB, Britton RA, Costa-Mattioli M. Mechanisms Underlying Microbial-Mediated Changes in Social Behavior in Mouse Models of Autism Spectrum Disorder. Neuron. 2019 Jan. 16;101(2):246-259. e6. doi: 10.1016/j.neuron.2018.11.018. Epub 2018 Dec. 3. PMID: 30522820; PMCID: PMC6645363.
11. Buffington SA, Dooling SW, Sgritta M, Noecker C, Murillo OD, Felice DF, Turnbaugh PJ, Costa-Mattioli M. Dissecting the contribution of host genetics and the microbiome in Cell. 2021 Apr. 1;184(7):1740-1756. e16. doi: 10.1016/j.cell.2021.02.009. Epub 2021 Mar. 10. PMID: 33705688.
12. Filippa M, Monaci MG, Spagnuolo C, Serravalle P, Daniele R, Grandjean D. Maternal speech decreases pain scores and increases oxytocin levels in preterm infants during painful procedures. Sci Rep. 2021 Aug. 27;11(1):17301. doi: 10.1038/s41598-021-96840-4. PMID: 34453088; PMCID: PMC8397753.
13. Hsiao EY, McBride SW, Hsien S, Sharon G, Hyde ER, McCue T, Codelli JA, Chow J, Reisman SE, Petrosino JF, Patterson PH, Mazmanian SK. Microbiota modulate behavioral and physiological abnormalities associated with neurodevelopmental disorders. Cell. 2013 Dec. 19;155(7):1451-63, doi: 10.1016/j.cell.2013.11.024. Epub 2013 Dec. 5. PMID: 24315484; PMCID: PMC3897394.
14. Buffington SA, Di Prisco GV, Auchtung TA, Ajami NJ, Petrosino JF, Costa-Mattioli M. Microbial Reconstitution Reverses Maternal Diet-Induced Social and Synaptic Deficits in Offspring. Cell. 2016 Jun. 16;165(7):1762-1775. doi: 10.1016/j.cell.2016.06.001. PMID: 27315483; PMCID: PMC5102250.
15. Needham BD, Funabashi M, Adame MD, Wang Z, Boktor JC, Hancy J, Wu WL, Rabut C, Ladinsky MS, Hwang SJ, Guo Y, Zhu Q, Griffiths JA, Knight R, Bjorkman PJ, Shapiro MG, Geschwind DH, Holschneider DP, Fischbach MA, Mazmanian SK. A gut-derived metabolite alters brain activity and anxiety behaviour in mice. Nature. 2022 February; 602(7898):647-653. doi: 10.1038/s41586-022-04396-8. Epub 2022 Feb. 14. PMID: 35165440.
16. Donaldson ZR, Young LJ. Oxytocin, vasopressin, and the neurogenetics of sociality. Science. 2008 Nov. 7;322(5903):900-4. doi: 10.1126/science.1158668. PMID: 18988842.
17. Dölen G, Darvishzadeh A, Huang KW, Malenka RC. Social reward requires coordinated activity of nucleus accumbens oxytocin and serotonin. Nature. 2013 Sep. 12;501(7466):179-84. doi: 10.1038/nature12518. PMID: 24025838; PMCID: PMC4091761.
18. Reichow B, Wolery M. Comprehensive synthesis of early intensive behavioral interventions for young children with autism based on the UCLA young autism project model. J Autism Dev Disord. 2009 January; 39(1):23-41. doi: 10.1007/s10803-008-0596-0. Epub 2008 Jun. 6. PMID: 18535894.
19. Charman T, Taylor E, Drew A, Cockerill H, Brown JA, Baird G. Outcome at 7 years of children diagnosed with autism at age 2: predictive validity of assessments conducted at 2 and 3 years of age and pattern of symptom change over time. J Child Psychol Psychiatry. 2005 May; 46(5):500-13. doi: 10.1111/j.1469-7610.2004.00377. x. PMID: 15845130.
20. Zwaigenbaum L, Bauman ML, Choueiri R, Kasari C, Carter A, Granpeesheh D, Mailloux Z, Smith Roley S, Wagner S, Fein D, Pierce K, Buie T, Davis PA, Newschaffer C, Robins D, Wetherby A, Stone WL, Yirmiya N, Estes A, Hansen RL, McPartland JC, Natowicz MR. Early Intervention for Children With Autism Spectrum Disorder Under 3 Years of Age: Recommendations for Practice and Research. Pediatrics. 2015 October; 136 Suppl 1:S60-81. doi: 10.1542/peds.2014-3667E. PMID: 26430170.
21. Gotham K, Pickles A, Lord C. Trajectories of autism severity in children using standardized ADOS scores. Pediatrics. 2012 November; 130(5):e1278-84. doi: 10.1542/peds.2011-3668. Epub 2012 Oct. 22. PMID: 23090336; PMCID: PMC3483889.
22. Farley MA, McMahon WM, Fombonne E, Jenson WR, Miller J, Gardner M, Block H, Pingree CB, Ritvo ER, Ritvo RA, Coon H. Twenty-year outcome for individuals with autism and average or near-average cognitive abilities. Autism Res. 2009 April; 2(2):109-18. doi: 10.1002/aur.69. PMID: 19455645.
23. Liu Y, Fatherce NY, Mangalat N, Rhoads JM. Human-derived probiotic Lactobacillus reuteri strains differentially reduce intestinal inflammation. Am J Physiol Gastrointest Liver Physiol. 2010 November; 299(5):G1087-96. doi: 10.1152/ajpgi.00124.2010. Epub 2010 Aug. 26. PMID: 20798357; PMCID: PMC2993169.
24. Spinler JK, Taweechotipatr M, Rognerud CL, Ou CN, Tumwasorn S, Versalovic J. Human-derived probiotic Lactobacillus reuteri demonstrate antimicrobial activities targeting diverse enteric bacterial pathogens. Anaerobe. 2008 June; 14(3):166-71. doi: 10.1016/j.anaerobe.2008.02.001. Epub 2008 Feb. 29. PMID: 18396068; PMCID: PMC2494590.
25. Walter J, Britton RA, Roos S. Host-microbial symbiosis in the vertebrate gastrointestinal tract and the Lactobacillus reuteri paradigm. Proc Natl Acad Sci U S A. 2011 Mar. 15;108 Suppl 1 (Suppl 1):4645-52. doi: 10.1073/pnas.1000099107. Epub 2010 Jun. 25. PMID: 20615995; PMCID: PMC3063604.
26. Duar RM, Frese SA, Lin XB, Fernando SC, Burkey TE, Tasseva G, Peterson DA, Blom J, Wenzel CQ, Szymanski CM, Walter J. Experimental Evaluation of Host Adaptation of Lactobacillus reuteri to Different Vertebrate Species. Appl Environ Microbiol. 2017 May 31;83(12):e00132-17. doi: 10.1128/AEM.00132-17. PMID: 28389535; PMCID: PMC5452824.
27. Garofoli F, Civardi E, Indrio F, Mazzucchelli I, Angelini M, Tinelli C, Stronati M. The early administration of Lactobacillus reuteri DSM 17938 controls regurgitation episodes in full-term breastfed infants. Int J Food Sci Nutr. 2014 August; 65(5):646-8. doi: 10.3109/09637486.2014.898251. Epub 2014 Mar. 17. PMID: 24635827.
28. Savino F, Garro M, Montanari P, Galliano I, Bergallo M. Crying Time and ROR6γ/FOXP3 Expression in Lactobacillus reuteri DSM17938-Treated Infants with Colic: A Randomized Trial. J Pediatr. 2018 January; 192:171-177. el. doi: 10.1016/j.jpeds.2017.08.062. Epub 2017 Sep. 29. PMID: 28969887.
29. Fatheree NY, Liu Y, Taylor CM, Hoang TK, Cai C, Rahbar MH, Hessabi M, Ferris M, McMurtry V, Wong C, Vu T, Dancsak T, Wang T, Gleason W, Bandla V, Navarro F, Tran DQ, Rhoads JM. Lactobacillus reuteri for Infants with Colic: A Double-Blind, Placebo-Controlled, Randomized Clinical Trial. J Pediatr. 2017 December; 191:170-178. e2. doi: 10.1016/j.jpeds.2017.07.036. Epub 2017 Sep. 29. PMID: 28969890; PMCID: PMC6336100.
30. Maya-Barrios A, Lira-Hernandez K, Jiménez-Escobar I, Hernández L, Ortiz-Hernandez A, Jiménez-Gutiérrez C, López-Velázquez G, Gutiérrez-Castellón P. Limosilactobacillus reuteri ATCC PTA 5289 and DSM 17938 as adjuvants to improve evolution of pharyngitis/tonsillitis in children: randomised controlled trial. Benef Microbes. 2021 Apr. 12;12(2):137-145. doi: 10.3920/BM2020.0171. Epub 2021 Apr. 1. PMID: 33789556.
31. Kong XJ, Liu K, Zhuang P, Tian R, Liu S, Clairmont C, Lin X, Sherman H, Zhu J, Wang Y, Fong M, Li A, Wang BK, Wang J, Yu Z, Shen C, Cui X, Cao H, Du T, Wan G, Cao X. The Effects of Limosilactobacillus reuteri LR-99 Supplementation on Body Mass Index, Social Communication, Fine Motor Function, and Gut Microbiome Composition in Individuals with Prader-Willi Syndrome: a Randomized Double-Blinded Placebo-Controlled Trial. Probiotics Antimicrob Proteins. 2021 December; 13(6):1508-1520. doi: 10.1007/s12602-021-09800 -9. Epub 2021 Jun. 11. PMID: 34115318; PMCID: PMC8578098.
32. Cassidy SB, Schwartz S, Miller JL, Driscoll DJ. Prader-Willi syndrome. Genet Med. 2012 January; 14(1):10-26. doi: 10.1038/gim.0b013e31822bead0. Epub 2011 Sep. 26. PMID: 22237428.
33. Bennett JA, Germani T, Haqq AM, Zwaigenbaum L. Autism spectrum disorder in Prader-Willi syndrome: A systematic review. Am J Med Genet A. 2015 December; 167A (12):2936-44. doi: 10.1002/ajmg.a.37286. Epub 2015 Aug. 29. PMID: 26331980.
34. Karren BC. A Test Review: Gilliam, J. E. (2014). Gilliam Autism Rating Scale-Third Edition (GARS-3). J Psychoeduc Assess. 2017,35(3):342-346. DOI: 10.1177/0734282916635465
35. Abdellatif B, Mcveigh C, Bendriss G, Chaari A. The Promising Role of Probiotics in Managing the Altered Gut in Autism Spectrum Disorders. Int J Mol Sci. 2020 Jun. 10;21(11):4159. doi: 10.3390/ijms21114159. PMID: 32532137; PMCID: PMC7312735.
36. Jovene MR, Bombace F, Maresca R, Sapone A, Iardino P, Picardi A, Marotta R, Schiraldi C, Siniscalco D, Serra N, de Magistris L, Bravaccio C. Intestinal Dysbiosis and Yeast Isolation in Stool of Subjects with Autism Spectrum Disorders. Mycopathologia. 2017 April; 182(3-4):349-363. doi: 10.1007/s11046-016-0068-6. Epub 2016 Sep. 21. PMID: 27655151.
37. Strati F, Cavalieri D, Albanese D, De Felice C, Donati C, Hayek J, Jousson O, Leoncini S, Renzi D, Calabrò A, De Filippo C. New evidences on the altered gut microbiota in autism spectrum disorders. Microbiome. 2017 Feb. 22;5(1):24. doi: 10.1186/s40168-017-0242-1. PMID: 28222761; PMCID: PMC5320696.
38. Kong XJ, Liu J, Liu K, Koh M, Sherman H, Liu S, Tian R, Sukijthamapan P, Wang J, Fong M, Xu L, Clairmont C, Jeong MS, Li A, Lopes M, Hagan V, Dutton T, Chan SP, Lee H, Kendall A, Kwong K, Song Y. Probiotic and Oxytocin Combination Therapy in Patients with Autism Spectrum Disorder: A Randomized, Double-Blinded, Placebo-Controlled Pilot Trial. Nutrients. 2021 May 5;13(5):1552. doi: 10.3390/nu13051552. PMID: 34062986; PMCID: PMC8147925.
39. Kong X, Liu J, Cetinbas M, Sadreyev R, Koh M, Huang H, Adeseye A, He P, Zhu J, Russell H, Hobbie C, Liu K, Onderdonk AB. New and Preliminary Evidence on Altered Oral and Gut Microbiota in Individuals with Autism Spectrum Disorder (ASD): Implications for ASD Diagnosis and Subtyping Based on Microbial Biomarkers. Nutrients. 2019 Sep. 6;11(9):2128. doi: 10.3390/nu11092128. PMID: 31489949; PMCID: PMC6770733.
40. Kong X, Liu J, Liu K, Koh M, Tian R, Hobbie C, Fong M, Chen Q, Zhao M, Budjan C, Kong J. Altered Autonomic Functions and Gut Microbiome in Individuals with Autism Spectrum Disorder (ASD): Implications for Assisting ASD Screening and Diagnosis. J Autism Dev Disord. 2021 January; 51(1):144-157. doi: 10.1007/s10803-020-04524-1. PMID: 32410097.
41. Kong XJ, Wan G, Tian R, Liu S, Liu K, Clairmont C, Lin X, Zhang X, Sherman H, Zhu J, Wang Y, Fong M, Li A, Wang BK, Wang J, Liu J, Yu Z, Shen C, Cui X, Cao H, Du T, Cao X. The Effects of Probiotic Supplementation on Anthropometric Growth and Gut Microbiota Composition in Patients With Prader-Willi Syndrome: A Randomized Double-Blinded Placebo-Controlled Trial. Front Nutr. 2021 Feb. 19;8:587974. doi: 10.3389/fnut.2021.587974. PMID: 33681271; PMCID: PMC7933553.
42. Huang M, Liu L, Liu K, Chen J, Wei Z, Feng Z, Wu Y, Fong M, Tian R, Wang B, Budjan C, Zhuang P, Wan G, Kong X-J. Microbiome-specific statistical modeling identifies interplay between gastrointestinal microbiome and neurobehavioral outcomes in patients with autism: a case control study. Front. Psychiatry. 2021 12:682454. Doi: 10.3389/fpsyt.2021.682454. PMID: 34744810
43. Cao X, Liu K, Liu J, Liu YW, Xu L, Wang H, Zhu Y, Wang P, Li Z, Wen J, Shen C, Li M, Nie Z, Kong XJ. Dysbiotic Gut Microbiota and Dysregulation of Cytokine Profile in Children and Teens With Autism Spectrum Disorder. Front Neurosci. 2021 Feb. 10;15:635925. doi: 10.3389/fnins.2021.635925. PMID: 33642989; PMCID: PMC7902875.
44. Smith, C. J.*, Kingsbury, M. A.*, Dziabis, J. E., Hanamsagar, R., Malcon, K. E., Tran, J. N., Morris, H. A., Gulino, M., Bordt, E. A. and Bilbo, S. D. (2020) Neonatal endotoxin challenge induces female-specific changes in social behavior and somatostatin cell number, independent of microglial-MyD88 signaling. Brain Behav. Immun, 90, 332-345. PMID: 32860938. * Indicates co-authorship.
45. Miranda-Ribera A, Serena G, Liu J, Fasano A, Kingsbury MA, Fiorentino MR. The Zonulin-transgenic mouse displays behavioral alterations ameliorated via depletion of the gut microbiota. Tissue Barriers. 2021 Nov. 14:2000299. doi: 10.1080/21688370.2021.2000299. Epub ahead of print. PMID: 34775911.
46. Singh A, Squires J, Yeh CJ, Heo KH, Bian H. Validity and reliability of the developmental assessment screening scale. J Family Med Prim Care. 2016 January-Mar; 5(1):124-8. doi: 10.4103/2249-4863.184636. PMID: 27453856; PMCID: PMC4943118.
47. Hardy S, Haisley L, Manning C, Fein D. Can Screening with the Ages and Stages Questionnaire Detect Autism? J Dev Behav Pediatr. 2015 September; 36(7):536-43. doi: 10.1097/DBP.0000000000000201. PMID: 26348972; PMCID: PMC4563819.
48. Beacham C, Reid M, Bradshaw J, Lambha M, Evans L, Gillespie S, Klaiman C, Richardson SS. Screening for Autism Spectrum Disorder: Profiles of Children Who Are Missed. J Dev Behav Pediatr. 2018 December; 39(9):673-682. doi: 10.1097/DBP.0000000000000607. PMID: 30059417.
49. Mullen E: Mullen Scales of Early Learning: AGS Edition. Circle Pines, MN: American Guidance Service; 1995.
50. Bums TG, King TZ, Spencer KS. Mullen scales of early learning: the utility in assessing children diagnosed with autism spectrum disorders, cerebral palsy, and epilepsy. Appl Neuropsychol Child. 2013;2(1):33-42. doi: 10.1080/21622965.2012.682852. Epub 2012 Apr. 26. PMID: 23427775.
51. Pandolfi V, Magyar CI. Vineland-3 Structural Validity and Interpretability of Domain Scores: Implications for Practitioners Assessing Adolescents With Developmental Conditions. Am J Intellect Dev Disabil. 2021 May 1;126(3):216-229. doi: 10.1352/1944-7558-126.3.216. PMID: 33910242.
52. Mclver LJ, Abu-Ali G, Franzosa EA, Schwager R, Morgan XC, Waldron L, Segata N, Huttenhower C. bioBakery: a meta'omic analysis environment. Bioinformatics. 2018 Apr. 1;34(7):1235-1237. doi: 10.1093/bioinformatics/btx754. PMID: 29194469; PMCID: PMC6030947.
53. Segata N, Izard J, Waldron L, Gevers D, Miropolsky L, Garrett WS, Huttenbower C. Metagenomic biomarker discovery and explanation. Genome Biol. 2011 Jun. 24;12(6): R60. doi: 10.1186/gb-2011-12-6-160. PMID: 21702898; PMCID: PMC3218848.
54. Higashida H, Yokoyama S, Huang JJ, Liu L, Ma WJ, Akther S, Higashida C, Kikuchi M, Minabe Y, Munesue T. Social memory, amnesia, and autism: brain oxytocin secretion is regulated by NAD+metabolites and single nucleotide polymorphisms of CD38. Neurochem Int. 2012 November; 61(6):828-38. doi: 10.1016/j.neuint.2012.01.030. Epub 2012 Feb. 13. PMID: 22366648.
55. LoParo D, Waldman ID. The oxytocin receptor gene (OXTR) is associated with autism spectrum disorder: a meta-analysis. Mol Psychiatry. 2015 May; 20(5):640-6. doi: 10.1038/mp.2014.77. Epub 2014 Aug. 5. PMID: 25092245.
56. Hörnberg H, Pérez-Garci E, Schreiner D, Hatstatt-BurkléL, Magara F, Baudouin S, Matter A, Nacro K, Pecho-Vrieseling E, Scheiffele P. Rescue of oxytocin response and social behaviour in a mouse model of autism. Nature. 2020 August; 584(7820):252-256. doi: 10.1038/s41586-020-2563-7. Epub 2020 Aug. 5. PMID: 32760004; PMCID: PMC7116741.
57. Mills M, Melhuish E. Recognition of mother's voice in early infancy. Nature. 1974 Nov. 8;252(5479):123-4. doi: 10.1038/252123a0. PMID: 4420642.
58. DeCasper AJ, Fifer WP. Of human bonding: newborns prefer their mothers' voices. Science. 1980 Jun. 6;208(4448):1174-6. doi: 10.1126/science.7375928. PMID: 7375928.
59. Mai X, Xu L, Li M, Shao J, Zhao Z, deRegnier RA, Nelson CA, Lozoff B. Auditory recognition memory in 2-month-old infants as assessed by event-related potentials. Dev Neuropsychol. 2012;37(5):400-14. doi: 10.1080/87565641.2011.650807. PMID: 22799760; PMCID: PMC3399741.
60. Deregnier RA, Nelson CA, Thomas KM, Wewerka S, Georgieff MK. Neurophysiologic evaluation of auditory recognition memory in healthy newborn infants and infants of diabetic mothers. J Pediatr. 2000 December; 137(6):777-84. doi: 10.1067/mpd.2000.109149. PMID: 11113833.
61. Eimas PD, Siqueland ER, Jusczyk P, Vigorito J. Speech perception in infants. Science. 1971 Jan. 22;171(3968):303-6. doi: 10.1126/science.171.3968.303. PMID: 5538846.
62. Gennari G, Marti S, Palu M, FlóA, Dehaene-Lambertz G. Orthogonal neural codes for speech in the infant brain. Proc Natl Acad Sci USA. 2021 Aug. 3;118(31): e2020410118. doi: 10.1073/pnas.2020410118. PMID: 34326247; PMCID: PMC8346888.
63. Seltzer L J, Ziegler T E, Pollak S D. Social vocalizations can release oxytocin in humans. Proc Biol Sci. 2010 Sep. 7;277(1694):2661-6. doi: 10.1098/rspb.2010.0567. Epub 2010May 12. PMID: 20462908; PMCID: PMC2982050.
64. Kanner, L. (1943) Autistic Disturbances of Affective Contact. Nervous Child, 2, 217-250.
65. Kanner L. Autistic disturbances of affective contact. Acta Paedopsychiatr. 1968;35(4):100-36. PMID: 4880460.
66. Klin A. Young autistic children's listening preferences in regard to speech: a possible characterization of the symptom of social withdrawal. J Autism Dev Disord. 1991 March; 21(1):29-42. doi: 10.1007/BF02206995. PMID: 1828067.
67. Abrams D A, Padmanabhan A, Chen T, Odriozola P, Baker A E, Kochalka J, Phillips J M, Menon V. Impaired voice processing in reward and salience circuits predicts social communication in children with autism. Elife. 2019 Feb. 26;8: e39906. doi: 10.7554/eLife.39906. PMID: 30806350; PMCID: PMC6391069.
68. Lloyd-Fox S, Blasi A, Pasco G, Gliga T, Jones E J H, Murphy D G M, Elwell C E, Charman T, Johnson M H; BASIS Team. Cortical responses before 6 months of life associate with later autism. Eur J Neurosci. 2018 March; 47(6):736-749. doi: 10.1111/ejn.13757. Epub 2017 Nov. 22. PMID: 29057543; PMCID: PMC5900943.
69. Gervais H, Belin P, Boddaert N, Leboyer M, Coez A, Sfaello I, Barthélémy C, Brunelle F, Samson Y, Zilbovicius M. Abnormal cortical voice processing in autism. Nat Neurosci. 2004 August; 7(8):801-2. doi: 10.1038/nn1291. Epub 2004 Jul. 18. PMID: 15258587.
70. Chen J, Liang C, Wei Z, Cui Z, Kong X, Dong C J, Lai Y, Peng Z, Wan G. Atypical longitudinal development of speech-evoked auditory brainstem response in preschool children with autism spectrum disorders. Autism Res. 2019 July; 12(7):1022-1031. doi: 10.1002/aur.2110. Epub 2019 Apr. 26. PMID: 31025832.
71. Schelinski S, Tabas A, von Kriegstein K. Altered processing of communication signals in the subcortical auditory sensory pathway in autism. Hum Brain Mapp. 2022 April 15;43(6):1955-1972. doi: 10.1002/hbm.25766. Epub 2022 Jan. 17. PMID: 35037743; PMCID: PMC8933247.
72. Riva V, Cantiani C, Mornati G, Gallo M, Villa L, Mani E, Saviozzi I, Marino C, Molteni M. Distinct ERP profiles for auditory processing in infants at-risk for autism and language impairment. Sci Rep. 2018 Jan. 15;8(1):715. doi: 10.1038/s41598-017-19009-y. PMID: 29335488; PMCID: PMC5768787.
73. Riva V, Riboldi EM, Dondena C, Piazza C, Molteni M, Cantiani C. Atypical ERP responses to audiovisual speech integration and sensory responsiveness in infants at risk for autism spectrum disorder. Infancy. 2022 March; 27(2):369-388. doi: 10.1111/infa.12456. Epub 2022 Jan. 17. PMID: 35037381.
74. Clairmont C, Wang J, Tariq S, Sherman HT, Zhao M, Kong XJ. The Value of Brain Imaging and Electrophysiological Testing for Early Screening of Autism Spectrum Disorder: A Systematic Review. Front Neurosci. 2022 Feb. 3;15:812946. doi: 10.3389/fnins.2021.812946. PMID: 35185452; PMCID: PMC8851356.
75. Chen J, Wei Z, Liang C, Liu B, Guo J, Kong X, Huang M, Peng Z, Wan G. Dysfunction of the Auditory Brainstem as a Neurophysiology Subtype of Autism Spectrum Disorder. Front Neurosci. 2021 Mar. 17;15:637079. doi: 10.3389/fnins.2021.637079. PMID: 33815042; PMCID: PMC8010248.
76. Huang Y, Yu S, Wilson G, Park J, Cheng M, Kong X, Lu T, Kong J. Altered Extended Locus Coeruleus and Ventral Tegmental Area Networks in Boys with Autism Spectrum Disorders: A Resting-State Functional Connectivity Study. Neuropsychiatr Dis Treat. 2021 Apr. 22;17:1207-1216. doi: 10.2147/NDT.S301106. PMID: 33911868; PMCID: PMC8075355.
77. Jung M, Tu Y, Lang CA, Ortiz A, Park J, Jorgenson K, Kong XJ, Kong J. Decreased structural connectivity and resting-state brain activity in the lateral occipital cortex is associated with social communication deficits in boys with autism spectrum disorder. Neuroimage. 2019 Apr. 15;190:205-212. doi: 10.1016/j.neuroimage.2017.09.031. Epub 2017 Sep. 18. PMID: 28927730.
78. Peng Z, Chen J, Jin L, Han H, Dong C, Guo Y, Kong X, Wan G, Wei Z. Social brain dysfunctionality in individuals with autism spectrum disorder and their first-degree relatives: An activation likelihood estimation meta-analysis. Psychiatry Res Neuroimaging. 2020 Apr. 30;298:111063. doi: 10.1016/j.pscychresns.2020.111063. Epub 2020 Mar. 5. PMID: 32179248.
79. Tian J, Wang J, Quana W, Lv B, Liu J, Hobbie C, Kong XJ, Dong W: The functional near-infrared spectroscopy in the diagnosis of schizophrenia. Eur. J. Psychiat. 2019;33(3):97-103. 2019 Jun. 14. https://doi.org/10.1016/j.ejpsy.2019.05.001
80. Huang M, Liu K, Wei Z, Feng Z, Chen J, Yang J, Zhong Q, Wan G, Kong XJ. Serum Oxytocin Level Correlates With Gut Microbiome Dysbiosis in Children With Autism Spectrum Disorder. Front Neurosci. 2021 Oct. 1;15:721884. doi: 10.3389/fnins.2021.721884. PMID: 34658767; PMCID: PMC8517432.
81. Kong XJ, Sherman HT, Tian R, Koh M, Liu S, Li AC, Stone WS. Validation of Rapid Interactive Screening Test for Autism in Toddlers Using Autism Diagnostic Observation Schedule™ Second Edition in Children at High-Risk for Autism Spectrum Disorder. Front Psychiatry. 2021 Oct. 1;12:737890. doi: 10.3389/fpsyt.2021.737890. PMID: 34658971; PMCID: PMC8517472.
82. Wan G\hat{ }, Kong X\hat{ }, Sun B, Yu S, Tu Y, Park J, Lang C, Koh M, Wei Z, Feng Z, Lin Y, Kong J. Applying Eye Tracking to Identify Autism Spectrum Disorder in Children. J Autism Dev Disord. 2019 January; 49(1):209-215. doi: 10.1007/s10803-018-3690-y. PMID: 30097760. \hat{ }co-first author
83. Kong XJ, Wei Z, Sun B, Tu Y, Huang Y, Cheng M, Yu S, Wilson G, Park J, Feng Z, Vangel M, Kong J and Wan G (2022) Different Eye Tracking Patterns in Autism Spectrum Disorder in Toddler and Preschool Children. Front. Psychiatry 13:899521. doi: 10.3389/fpsyt.2022.899521
84. Stavropoulos KK, Carver LJ. Neural Correlates of Attention to Human-Made Sounds: An ERP Study. PLOS One. 2016 Oct. 31;11(10): e0165745. doi: 10.1371/journal.pone.0165745. PMID: 27798701; PMCID: PMC5087949.
85. Kuhn P, Zores C, Pebayle T, Hoeft A, Langlet C, Escande B, Astruc D, Dufour A. Infants born very preterm react to variations of the acoustic environment in their incubator from a minimum signal-to-noise ratio threshold of 5 to 10 dBA. Pediatr Res. 2012 April; 71(4 Pt 1):386-92. doi: 10.1038/pr.2011.76. Epub 2012 Feb. 15. PMID: 22391640.
86. White R D, Smith J A, Shepley M M; Committee to Establish Recommended Standards for Newborn ICU Design. Recommended standards for newborn ICU design, eighth edition. J Perinatol. 2013 April; 33 Suppl 1: S2-16. doi: 10.1038/jp.2013.10. PMID: 23536026.
87. Shultz S, Klin A, Jones W. Inhibition of eye blinking reveals subjective perceptions of stimulus salience. Proc Natl Acad Sci U S A. 2011 Dec. 27;108(52):21270-5. doi: 10.1073/pnas. 1109304108. Epub 2011 Dec. 12. PMID: 22160686; PMCID: PMC3248475.
88. Speer L L, Cook A E, McMahon W M, Clark E. Face processing in children with autism: effects of stimulus contents and type. Autism. 2007 May; 11(3):265-77. doi: 10.1177/1362361307076925.
89. Jones W, Klin A. Attention to eyes is present but in decline in 2-6-month-old infants later diagnosed with autism. Nature. 2013 Dec. 19;504(7480):427-31. doi: 10.1038/nature12715. Epub 2013 Nov. 6. PMID: 24196715; PMCID: PMC4035120.
90. Tamura A, Iwamoto T, Ozaki H, Kimura M, Tsujimoto Y, Wada Y. Wrist-Worn Electrodermal Activity as a Novel Neurophysiological Biomarker of Autonomic Symptoms in Spatial Disorientation. Front Neurol. 2018 Dec. 4;9:1056. doi: 10.3389/fneur.2018.01056. PMID: 30564188; PMCID: PMC6288226.
91. Kushki A, Brian J, Dupuis A, Anagnostou E. Functional autonomic nervous system profile in children with autism spectrum disorder. Mol Autism. 2014 Jul. 4;5:39. doi: 10.1186/2040-2392-5-39. PMID: 25031832; PMCID: PMC4099494.
92. Wagner J B, Luyster R J, Tager-Flusberg H, Nelson C A. Greater Pupil Size in Response to Emotional Faces as an Early Marker of Social-Communicative Difficulties in Infants at High Risk for Autism. Infancy. 2016 September-Oct; 21(5):560-581. doi: 10.1111/infa. 12128. Epub 2016 Feb. 4. PMID: 27616938; PMCID: PMC5016034.
93. Kommers D R, Broeren M, Andriessen P, Oei SG, Feijs L, Bambang Oetomo S. Pilot study demonstrates that salivary oxytocin can be measured unobtrusively in preterm infants. Acta Paediatr. 2017 January,106(1):34-42. doi: 10.1111/apa.13606. Epub 2016 Oct. 17. PMID: 27666329; PMCID: PMC5215538.
94. Tomchek S D, Dunn W. Sensory processing in children with and without autism: a comparative study using the short sensory profile. Am J Occup Ther. 2007 March-Apr; 61(2):190-200. doi: 10.5014/ajot.61.2.190. PMID: 17436841.
95. Theofanopoulou C. Implications of Oxytocin in Human Linguistic Cognition: From Genome to Phenome. Front Neurosci. 2016 Jun. 14;10:271. doi: 10.3389/fnins.2016.00271. PMID: 27378840; PMCID: PMC4906233.
96. Higashida H, Yokoyama S, Huang J J, Liu L, Ma W J, Akther S, Higashida C, Kikuchi M, Minabe Y, Munesue T. Social memory, amnesia, and autism: brain oxytocin secretion is regulated by NAD+ metabolites and single nucleotide polymorphisms of CD38. Neurochem Int. 2012 November; 61(6):828-38. doi: 10.1016/j.neuint.2012.01.030. Epub 2012 Feb. 13. PMID: 22366648.
97. Kitagawa K, Matsumura K, Baba M, Kondo M, Takemoto T, Nagayasu K, Ago Y, Seiriki K, Hayata-Takano A, Kasai A, Takuma K, Hashimoto R, Hashimoto H, Nakazawa T. Intranasal oxytocin administration ameliorates social behavioral deficits in a POGZWT/Q1038R mouse model of autism spectrum disorder. Mol Brain. 2021 Mar. 16;14(1):56. doi: 10.1186/s13041-021-00769-8. PMID: 33726803; PMCID: PMC7962304.
98, Sikich L, Kolevzon A, King BH, McDougle CJ, Sanders KB, Kim SJ, Spanos M, Chandrasekhar T, Trelles MDP, Rockhill CM, Palumbo ML, Witters Cundiff A, Montgomery A, Siper P, Minjarez M, Nowinski LA, Marler S, Shuffrey LC, Alderman C, Weissman J, Zappone B, Mullett JE, Crosson H, Hong N, Siecinski SK, Giamberardino SN, Luo S, She L, Bhapkar M, Dean R, Scheer A, Johnson JL, Gregory SG, Veenstra-VanderWeele J. Intranasal Oxytocin in Children and Adolescents with Autism Spectrum Disorder. N Engl J Med. 2021 Oct. 14;385(16):1462-1473. doi: 10.1056/NEJMoa2103583. PMID: 34644471. Enrolled 290 children with ASD.
99. Dadds MR, MacDonald E, Cauchi A, Williams K, Levy F, Brennan J. Nasal oxytocin for social deficits in childhood autism: a randomized controlled trial. J Autism Dev Disord. 2014 March; 44(3):521-31. doi: 10.1007/s10803-013-1899-3. PMID: 23888359.

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

All methods described herein can be performed in any suitable order unless otherwise indicated herein or otherwise clearly contradicted by context. The use of any and all examples provided herein, is intended merely to better illuminate the invention and does not pose a limitation on the scope of the invention unless otherwise claimed. No language in the specification should be construed as indicating any non-claimed element as essential to the practice of the invention.

Citations to a number of patent and non-patent references are made herein. The cited references are incorporated by reference herein in their entireties. In the event that there is an inconsistency between a definition of a term in the specification as compared to a definition of the term in a cited reference, the term should be interpreted based on the definition in the specification.

We claim:

1. A method for assessing a risk of a subject developing autism spectrum disorder (ASD), comprising:
determining a level of Limosilactobacillus reuteri (L. reuteri) in a sample from the subject, or receiving results of a test indicating a level of L. reuteri in the sample from the subject; and
determining the risk of developing ASD in the subject, based at least partly on the level of L. reuteri in the sample from the subject.

2. The method of claim 1, wherein subject is an infant.

3. The method of claim 1, wherein an age of the subject is from about one day to about three years.

4. The method of claim 1, wherein the level of L. reuteri in the sample from the subject is determined using quantitative Polymerase Chain Reaction (qPCR).

5. The method of claim 4, wherein the qPCR utilizes a first primer comprising the polynucleotide sequence of SEQ ID NO: 1, and a second primer comprising the polynucleotide sequence of SEQ ID NO: 2.

6. The method of claim 1, wherein the determining the risk of developing ASD in the subject comprises comparing the level of L. reuteri in the sample from the subject to a threshold value.

7. The method of claim 1, wherein the sample from the subject is a stool sample.

8. The method of claim 1, further comprising:
determining responsiveness to human vocal sounds in the subject.

9. The method of claim 8, wherein determining responsiveness to human vocal sounds includes determining a level of oxytocin in a saliva sample from the subject, recording event-related potentials (ERP) in the subject.

10. The method of claim 8, wherein the determining the risk of developing ASD in the subject, is based at least partly on the level of L. reuteri in the sample from the subject and the responsiveness to human vocal sounds in the subject.

11. The method of claim 1, further comprising: prescribing or performing a therapeutic intervention designed to reduce the onset of one or more symptoms associated with ASD.

12. The method of claim 11, wherein the therapeutic intervention comprises administering to the subject L. reuteri.

13. The method of claim 12, wherein the L. reuteri administered to the subject is L. reuteri ATCC PTA 6475.

14. The method of claim 12, wherein the L. reuteri is orally administered to the subject.

15. The method of claim 12, further comprising, subsequent to the administering to the subject L. reuteri, determining responsiveness to human vocal sounds in the subject.

16. The method of claim 1, further comprising determining a level of tetrahydrobiopterin (BH4) in the sample from the subject.

17. The method of claim 16, wherein the level of BH4 in the sample from the subject is determined using an enzyme-linked immunosorbent assay (ELISA).

18. A kit for assessing a risk of a subject developing autism spectrum disorder (ASD) wherein the subject has an age of from about one day to three years, comprising:
one or more reagents for determining a level of L. reuteri in a sample from the subject; and
instructions for determining the level of L. reuteri in the sample from the subject.

19. The kit of claim 18, wherein the one or more reagents comprise a pair of primers for quantitative Polymerase Chain Reaction (qPCR), and wherein a first primer of the pair of primers comprises the polynucleotide sequence of SEQ ID NO: 1, and wherein a second primer of the pair of primers comprises the polynucleotide sequence of SEQ ID NO: 2.

20. The kit of claim 18, wherein the instructions for determining the level of L. reuteri comprise instructions for determining the level of L. reuteri in the sample from the subject, based on results of the qPCR of the sample and on the results of a control sample.
